(19) United States
(12) Patent Application Publication
(10) Pub. No.: US 2022/0409581 A1
(43) Pub. Date: Dec. 29, 2022

(54) METHODS AND COMPOSITIONS FOR TREATMENT OF SOLID TUMORS USING F16 ISOINDOLE SMALL MOLECULES

(71) Applicant: Nova Southeastern University, Fort Lauderdale, FL (US)

(72) Inventors: Appu RATHINAVELU, Fort Lauderdale, FL (US); Thiagarajan VENKATESAN, Fort Lauderdale, FL (US); Jayanta K. DAS, Fort Lauderdale, FL (US); Mohammad Mansour S. ALGAHTANI, Fort Lauderdale, FL (US)

(73) Assignee: Nova Southeastern University, Fort Lauderdale, FL (US)

(21) Appl. No.: 17/776,273

(22) PCT Filed: Nov. 13, 2020
(86) PCT No.: PCT/US2020/060549

§ 371 Date: May 12, 2022

Related U.S. Application Data
(60) Provisional application No. 62/935,448, filed on Nov. 14, 2019.

(51) Int. Cl. A61K 31/4035; A61K 31/495; A61K 39/395; A61P 35/00

(52) U.S. Cl. CPC A61K 31/4035; A61K 31/495; A61K 39/3955; A61P 35/00

(57) ABSTRACT

The invention provides methods for using F16 isoindole small molecules for treatment of solid tumors, particularly brain cancers, such as glioblastoma multiforme (GBM). F16 isoindole is an inhibitor of angiogenesis and is capable of antagonizing tumor vasculature. The invention also provides pharmaceutical compositions including F16 isoindole small molecules.

### FIELD OF THE INVENTION

The invention is encompassed within the field of cancer therapy and generally relates to therapies using small molecules to target solid tumors, particularly to therapies using F16 isoindole small molecules for treatment of brain cancers.

### BACKGROUND

Despite the significant efforts and resources that are being devoted for developing newer treatment strategies and cures, cancer remains a fatal disease of mankind and millions of people around the world die every year from various types of cancers. One of the most prevailing types of this deadly disease is brain cancer, which is the leading cause of cancer-related deaths in children and the third most common cause of cancer related death in adolescents and young adults between age 15 and 39 years [1, 2].

There are 12 main groups of brain tumors and more than 100 subgroups that share common biological features [3]. Gliomas include all tumors arising from the supportive tissues of the brain and are the most aggressive form of brain cancers, account for 24.7% of all primary brain tumors, and 74.6% of all malignant brain tumors [4]. Glioblastoma multiforme (GBM) is the most diagnosed form of glioma in the United States and is the most lethal type worldwide. Despite use of multidisciplinary treatment approaches, GBM has a very low 5-year survival rate of 5%, and a median survival of about one year post-diagnosis [3, 4]. Generally, GBM is classified as a grade IV glioma, and some of the histologic features that distinguish it from other grades are the presence of necrosis and the dramatic increase of blood vessel growth around the tumor [5]. In fact, GBM is one of the most highly vascularized solid tumors since its growth depends on angiogenesis as supported by various preclinical studies that have indicated that the glioma growth critically depends on the generation of tumor-associated blood vessels [4, 6]. In addition, GBM tumor vasculature is characterized by a dense network of vessels that are tortuous and hyper-permeable and have abnormally increased vessel diameter and thickness of basement membranes. This aberrant tumor vasculature is believed to enhance tumor hypoxia and impair the delivery of cytotoxic chemotherapy thus contributing to the treatment failure [5, 7]. Therefore, antagonizing tumor vascular is emerging as a novel strategy for brain tumor treatment, particularly for treatment of GBM.

Several forms of treatments that are currently available for GBM are non-effective in many cases, therefore, prognosis for GBM remains poor. The current treatment of glioblastoma involves surgery, whenever it is applicable, followed by radiation and chemotherapy with Temozolomide (TMZ). This treatment strategy provides a modest increase in overall survival [8]. In preclinical and clinical studies, the use of angiogenesis inhibitors in combination with chemotherapeutic agents has shown promising results against a wide range of cancer types [9-12]. Particularly, antiangiogenic agents are currently under intense investigation for treating GBM and various preliminary studies have yielded promising outcomes [13-15]. Therefore, several antiangiogenic agents are now in clinical trials for the treatment of GBM in monotherapy or in combination [16]. So far, Bevacizumab (BVZ), a monoclonal antibody with anti-angiogenic effects, has been approved by FDA for the treatment of recurrent GBM. The FDA approval of BVZ was based on an increase in the overall Objective Response Rate (ORR). However, in depth analysis of BVZ treatment data of patients with GBM showed no improvement in overall survival (OS) [17, 18]. It is worth mentioning that angiogenesis inhibitors, when used in monotherapy, can generally produce cytostatic effect and maximum therapeutic efficacy is achieved if these agents are combined with cytotoxic chemotherapeutic agents [19, 20].

One of the major obstacles of treating a brain tumor is the ability of the therapeutic agent to cross the blood-brain barrier (BBB) [21]. It is well known that penetrating BBB is not easy for agents with high molecular weight such as BVZ (˜150 kD M.W.), implying that BVZ treatment for GBM may not provide optimal delivery and treatment outcomes [22, 23]. Therefore, recent interest has shifted towards exploring small molecules that can cross BBB to modulate angiogenesis and similar processes. In this context, a novel compound, isoindole (1, 3-dioxy-2, 3-dihydro-1H-isoindol-4-yl)-amide, was developed at Nova Southeastern University (NSU) and code named as F16. See U.S. Pat. No. 7,875,603; Japanese Patent 5436544; and Korean Patent 10-1538822. F16 chemical structure (19, references of Example 2):

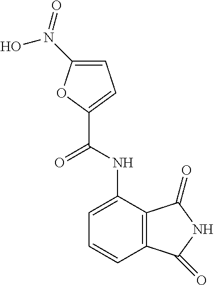

F16 is not only showing strong vascular endothelial growth factor receptor-2 (VEGFR-2) binding and inhibition of VEGFR-2 phosphorylation in human umbilical vein endothelial cells (HUVEC) but F16 also exhibits a significant in vivo tumor growth inhibition in mice implanted with breast and [24] colorectal cancer xenografts (data not published). More importantly, the preclinical pharmacokinetics studies have shown that F16 can cross BBB and accumulate into brain regions [25]. Furthermore, results from preclinical safety studies have proven so far that F16 treated experimental animals remain healthy compared to the groups that were treated with other FDA approved anticancer agents such as Paclitaxel [24] and Sunitinib [25].

The small molecule F16 (isoindole) exerts antiangiogenic effects by blocking vascular endothelial growth factor receptor 2 (VEGFR2), which is necessary for the development of new blood vessels in a solid tumor such as breast cancer (FIG. 1). In studies conducted at the Rumbaugh-Goodwin Institute for Cancer Research, the patented small molecule F16 has demonstrated both anti-angiogenic and pro-apoptotic (programmed cell death) effects against solid tumors. This novel compound showed promising anticancer effects in both cell culture and in vivo experiments and demonstrated relatively less toxicity when compared with some of the existing, FDA approved anticancer drugs. Studies on a breast cancer xenograft mouse model indicated that F16 has significant anticancer effects, owing to its anti-angiogenic properties and its tumor inhibitory abilities, which were comparable to a commonly used chemotherapeutic agent, Paclitaxel (Taxol). Moreover, in this mouse model study, F16 exhibited much less toxicity compared to Taxol. In xenograft studies also, F16 proved to be efficient in inhibiting tumor growth when used by itself or as a combination therapy with Paclitaxel. When both F16 and Taxol were used in combination treatments in an in vivo study, it not only resulted in nearly 85% suppression of tumor growth but also did not produce significant toxicity that is often associated with Taxol monotherapy. The findings of these studies offered substantial evidence supporting the use of F16 as anticancer agents for treating cancers with angiogenic abilities. In addition to treatment studies with the subcutaneously implanted xenograft treatments, tissue distribution of F16 was analyzed, which showed accumulation in the brain in the range of 5000 ng/g of tissue (FIG. 2). This study prompted the instant inventors with the idea that F16 could be useful for the treatment of brain cancers which show angiogenic ability for their survival and growth.

Since the inventive methods (and compositions) described herein show the efficacy of F16 in delaying glioblastoma progression via its anti-angiogenic and pro-apoptotic abilities, it (F16) can potentially be used as a basis to create new avenues for effective treatment of brain cancers, particularly those exhibiting angiogenic ability which enables their growth and survival.

### SUMMARY OF THE INVENTION

The small molecule F16 (isoindole) offers the potential for promising new cancer therapy. Based on preliminary in vitro and in vivo experiments, the cytotoxic effects in the monolayer culture and in 3D culture were confirmed. To achieve a good understanding about the effects of F16 on the migration and invasive ability of cancer cells, a Scratch assay, a Trans-well migration assay, and an invasion assay were performed. The anti-migratory effects and the anti-invasive capacity of the U87MG cells, that typically coincide with the anti-angiogenic properties during cancer metastasis, were determined through the above-mentioned assays. The results confirmed that invading abilities of U87MG cells were significantly decreased after 24 h of treatment with F16 versus untreated control cells in a dose-dependent manner. The results were compared with TMZ (Temozolomide) which is an FDA approved drug. So far, F16 has shown consistent inhibitory effects on the cell migration as well as cancer cell invasion, as presented in the results, which are significantly better than the TMZ effects. The changes in the pro-apoptotic gene expressions were also analyzed and it appeared that F16 can inhibit cell cycle and induce apoptosis in the U87MG cell line better than the TMZ.

The luciferase gene transfected U87MG-luc tumor cells were established, to monitor the tumor growth inhibition through optical imaging while assessing the effectiveness of F16 for the treatment of glioblastoma. Initially, the xenograft model was created by injecting the U87MG-Luc cells using intra-peritoneal injection (i.p.). The animals were treated with F16, TMZ, and a combination of both drugs. The studies with U87MG-Luc glioblastoma cell line have shown good results with the F16 compound. While reducing the tumor volume, F16 did not alter the body weight during the treatment period. Analysis of the blood parameters such as RBC, WBC (FIGS. 14A-B), Hemoglobin levels, Hematocrit etc. (FIGS. 14C-G) in F16 treated animals showed no signs of toxicity. While looking at the marker of liver during blood chemistry analysis, TMZ was significantly elevating the ALT (Alanine Transaminase) levels while it remained close to normal in the F16 treated cells. Both F16 and JFD showed no elevation of BUN (blood urea nitrogen) levels, which suggested that the kidney function was not affected by both drugs. Similarly, blood glucose, calcium, phosphorous and protein levels remained within the normal range (FIGS. 14C-G).

After completing testing, the effect with the subcutaneous tumor models, and after confirming the safety of F16, the intracranial implant studies were initiated. In the intracranial experiments, F16 was able to block the tumor growth of the brain in 50% of the animals. This conformed that F16 was able to cross the BBB and inhibit the growth of the U87MG derived tumor in the brain. It has been also noted that KP (Kolliphor®) that was used as a vehicle was slightly increasing the brain delivery of F16 but was also causing some side effects.

In a most basic aspect, the invention provides methods for manipulation of malignant cells, particularly interactions within malignant cells characterized by uncontrolled growth.

In another basic aspect, the invention provides a new treatment modality for cancer.

In a general aspect, the invention provides methods and compositions for treatment of cancers manifesting as solid tumors, particularly, but not limited to, solid tumors exhibiting angiogenic ability.

In a general aspect, the invention provides methods and compositions for treatment of cancer, particularly, but not limited to, brain cancers, such as gliomas.

In an aspect, the invention provides methods and compositions for treatment of aggressive and/or late stage brain cancer, particularly, but not limited to, glioblastoma multiforme (GBM).

In an aspect, the invention provides compositions for treatment of solid tumors having angiogenic ability and/or brain cancer, particularly, but not limited to, GBM, (the compositions) including F16 (isoindole) small molecules. The terms “F16” and “isoindole” are used interchangeably herein.

In another aspect, the invention provides pharmaceutical compositions for treatment of solid tumors and/or brain cancer, particularly, but not limited to, GBM, (the pharmaceutical compositions) including a therapeutically effective dosage of F16 in a pharmaceutical carrier. The “pharmaceutical carrier” can be any inactive and non-toxic agent useful for preparation of medications. The phrase “therapeutically effective dosage” or “therapeutically effective amount” refers to the amount of a composition required to achieve the desired function; for example, inhibition of vascular endothelial growth factor receptor-2 (VEGFR-2) in malignant cells. Malignant cells are cells characterized by uncontrolled growth. The terms “malignant cells”, “cancer cells,” and “tumor cells” are used interchangeably herein.

In an aspect, in addition to the therapeutically effective dosage of F16, the pharmaceutical composition can include a therapeutically effective dosage of a chemotherapeutic agent, particularly, but not limited to, temozolomide (TMZ) or bevacizumab (BVZ) or similar agents.

In an aspect, the invention provides various methods of using F16 compositions for treating malignant cells, such as, but not limited to, malignant cells of a brain cancer. These methods include steps of providing the F16 compositions described herein and administering the compositions to the malignant cells. These methods include, but are not limited to, inhibiting VEGFR-2 in malignant cells, inhibiting phosphorylation of VEGFR-2 in malignant cells, inhibiting migration and invasion of malignant cells into surrounding tissues, inhibiting a cell cycle in malignant cells, arresting a cell cycle in malignant cells, and inducing apoptosis in malignant cells.

In another aspect, the invention provides a method for inhibiting and/or arresting angiogenesis in tissue exhibiting aberrant vasculature. This method includes the steps of providing the F16 compositions described herein and administering the compositions to the tissue exhibiting aberrant vasculature. This method can be used as a treatment for highly vascular solid tumors or for any tumor having the ability to produce new blood vessels. A non-limiting example of such a tumor is brain cancer.

In yet another aspect, the invention provides a method for treating glioblastoma multiforme (GBM) in a subject in need thereof. This method includes the steps of providing the F16 compositions described herein and administering the compositions to the subject. The term “subject” refers to any human or animal who will benefit from the use of the compositions, methods, and/or treatments described herein. A preferred, but non-limiting example of a subject is a human patient having brain cancer.

Other objectives and advantages of this invention will become apparent from the following description, wherein are set forth, by way of example, certain embodiments of this invention.

### BRIEF DESCRIPTION OF THE DRAWINGS

A more complete understanding of the present invention may be obtained by references to the accompanying drawings when considered in conjunction with the subsequent detailed description. The embodiments illustrated in the drawings are intended only to exemplify the invention and should not be construed as limiting the invention to the illustrated embodiments.

FIG. 1 is a schematic illustration of the mechanism of F16 binding to the vascular endothelial growth factor receptor-2 (VEGFR2) which binding prevents binding of vascular endothelial growth factor (VEGF) to the receptor thus achieving an anti-angiogenic effect.

FIG. 2 is a bar graph showing tissue distribution of F16.

FIGS. 3A-C are graphs showing results of cytotoxicity assays. Cell viability was assessed using a 3-(4,5-dimethylthiazol-2-yl)-2, 5-diphenyltetrazolium bromide (MTT) assay (Sigma-Aldrich, St. Louis, Mo., USA) and a trypan blue dye exclusion method (TBDE). IC ₅₀ is the concentration of a drug that is required for 50% inhibition.

FIGS. 4A-B are images showing morphology of the U87MG cells during treatment with F16 or TMZ (prior to cell death).

FIGS. 5A-D show migration ability of U87MG cells using a scratch assay.

FIGS. 6A-D show migration ability of U87MG cells using a trans-well assay.

FIGS. 7A-D show invasive ability of U87MG cells using a cell invasion assay.

FIGS. 8A-B show effect of F16, TMZ, and combinations on anchorage-independent growth of U87MG cells using a soft agar colony formation assay.

FIG. 9 shows gene expression in U87MG cells using reverse transcription polymerase chain reaction (RT-PCR) analysis.

FIGS. 10A-C show protein expression in U87MG cells using a western blot analysis.

FIGS. 11A-E shows results obtained from development of a glioblastoma xenograft animal model.

FIGS. 12A-B show results from selection and measurement of Luciferase signal in U87MG-Luc cells.

FIG. 13 is bar graph documenting body weight change in the mice.

FIGS. 14A-G are bar graphs showing the hematological parameters of the mice.

FIG. 15 shows Table 1, which references the hematological parameters of the mice.

FIGS. 16A-H are bar graphs showing the biochemical parameters of the mice.

FIG. 17 shows Table 2, which references the biochemical parameters of the mice.

FIGS. 18A-D show data evidencing inhibition of U87MG-derived xenograft tumor growth by F16 in the mice.

FIGS. 19A-B show survival rate (of the mice) and signs of toxicity (in the mice).

FIGS. 20A-F are images showing results of a microvessel density assessment.

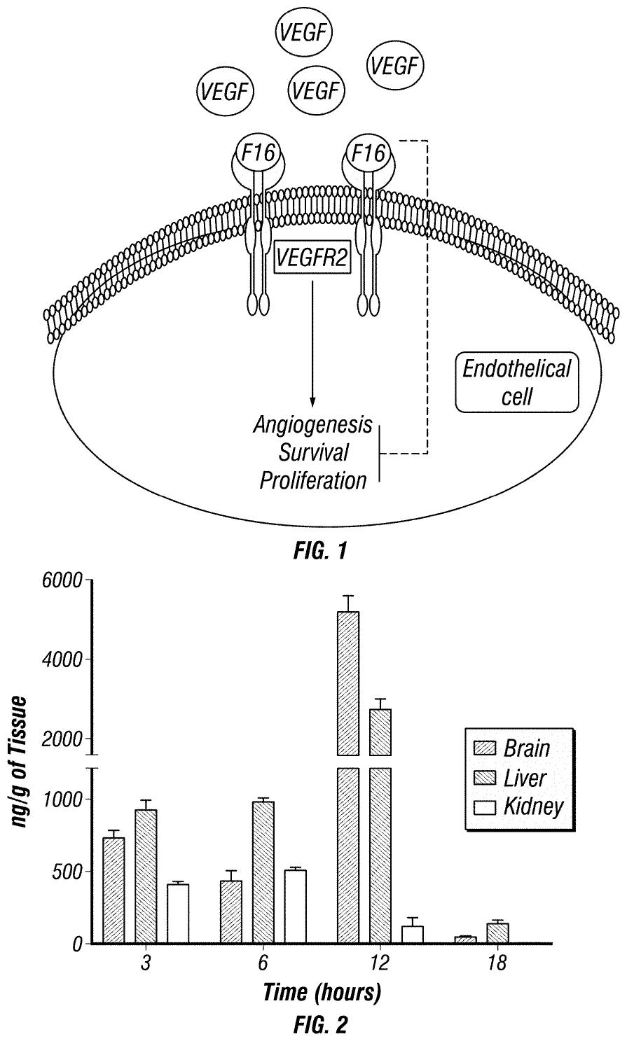

### DETAILED DESCRIPTION OF THE INVENTION

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described compositions and methods along with any further application of the principles of the invention as described herein, are contemplated as would normally occur to one skilled in the art to which the invention relates.

Glioblastoma multiforme (GBM) is one of the most aggressive and lethal types of cancer having an exceptionally low 5-year survival rate. Therefore, development of effective treatment for GBM is urgently desired. Since GBM is a highly vascularized tumor and its growth is angiogenesis-dependent, antagonizing tumor angiogenesis by using angiogenesis inhibitors seems to be a promising approach that is undergoing various stages of evaluation. In this context, intensive preclinical evaluation of F16, a novel small molecule, has exhibited potent anti-angiogenic and anti-tumor activities via selectively antagonizing vascular endothelial growth factor receptor-2 (VEGFR-2). More importantly, pharmacokinetic evaluation with tissue distribution analysis of F16 showed that F16 transported across the blood brain barrier (BBB) and accumulated into the brain regions with no signs of neurotoxicity. Therefore, further studies were conducted to determine the efficacy of F16 in delaying glioblastoma progression via inhibiting tumor angiogenesis. In vitro studies have clearly demonstrated inhibition of migration and invasion of U87MG cells and confirmed a potent cytotoxic effect against these cells in comparison to TMZ (IC₅₀ 26 μM vs 430 μM). In addition, F16 inhibited the VEGF receptor via competitive binding and blocked the phosphorylation of VEGFR-2, to induce cell cycle arrest and apoptosis by activating p53 mediated pathway. Furthermore, in vivo studies with the subcutaneously implanted (s.c.) xenograft model indicated that F16 treatment is efficacious in delaying tumor growth. So far, results suggest that F16 treatment could effectively induce cell cycle arrest and cause tumor reductive effect. F16 can also cross the BBB to reach the brain and therefore is emerging as a viable agent for targeting glioblastoma.

### Example 1: Xenograft Model of Glioblastoma

### Material and Methods

### Cell Line and Reagents

U87MG, a human glioblastoma cell line, was purchased from ATCC (Manassas, Va., USA) and maintained in Eagle's minimum essential medium (EMEM) supplemented with 10% fetal bovine serum, 2 mM L-glutamine, 1.5 g/L sodium bicarbonate and 1% penicillin/streptomycin. Cells were incubated at 37° C. with 95% air and 5% CO₂ in a humidified incubator. U87MG cells were used in assays, when the cell passages were between 3 and 9. The F16 and TMZ (Sigma-Aldrich, St. Louis, Mo., USA) were prepared as a solution in dimethyl sulfoxide (DMSO). The antibodies against VEGFR-2, p-VEGFR-2 (Tyr 1175), AKT, p-AKT (Ser473), ERK1/2, p-ERK1/2, p53, p21, Bax, Bcl2, MMP-2 and MMP-9 were purchased from Cell Signaling Technology (Danvers, Mass., USA). All other chemicals used in these experiments were of research grade.

### Cytotoxicity Assay

The cell viability was assessed using a 3-(4,5-dimethylthiazol-2-yl)-2, 5-diphenyltetrazolium bromide (MTT) assay (Sigma-Aldrich, St. Louis, Mo., USA), and trypan blue dye exclusion method (TBDE). For the MTT assay, U87MG cells were cultured in a 96 well-plate at a density of 5×10³ per well and incubated at 37° C. under 5% CO₂ for 24 h. Then, the cells were treated with different concentrations of F16 (0.1-100 μM) and TMZ (0.1-500 μM) for 24 h. At the end of treatment, the old medium was aspirated, and 10 μL of MTT (0.5 mg/mL in PBS) was added to each well and the cells were incubated at 37° C. for an additional 3 h. Finally, the MTT solution was removed, and 100 μL of dimethyl sulfoxide (DMSO) was added to each well. The plate was gently rotated on an orbital shaker for 10 min to completely dissolve the precipitation and the absorbance was measured at 570 nm using Microplate Reader (VersaMax, Molecular Devices, Sunnyvale, Calif., USA). For the TBDE method, U87MG cells were cultured in a 24 well-plate at a density of 5×10⁴ per well and incubated at 37° C. under 5% CO₂ for 48 h. Then, the cells were treated with different concentrations of F16 (0.1-100 μM) and TMZ (10-1000 μM). After 24, 48, and 72 h of treatments, an aliquot (50 μL) of the cell suspension from each treatment was mixed with 1:1 (v/v) volume of 0.4% trypan blue. The viable cells were counted with a Bio-Rad TC20™ Automated Cell Counter (Hercules, Calif., USA)

### Morphological Observation

U87MG cells were grown to 70%-80% confluence in 6-well culture plates. Then various concentrations of F16 (0.1-100 μM) and TMZ (10-1000 μM) were added to the media. After 24 h of treatments, morphological changes were documented with a Leica microscope (100× magnification). At least, 3 vision fields from each treatment wells were captured to see the changes in the cell morphology.

### Migration Assay

The migration ability of U87MG cell was determined using both scratch and trans-well assays. For the scratch assay, monolayer of U87MG cells was grown on 6-well plates close to 80% confluency. Using a sterile 200 μL tip, a single straight line scratch was made in each well. The wells were washed with phosphate-buffered saline (PBS) and refilled with growth medium containing various concentrations of F16 (0.1-20 μM) and TMZ (10-400 μM). The images were captured using Leica microscope at 12 h and 24 h post-scratch. For the trans-well migration assay, 6.5 mm trans-well plates polycarbonate membrane inserts (Corning, N.Y., USA) with 8 μm pore size were used. After an initial equilibrium period, 5×10⁴ cells suspended in 100 μL basal medium without FBS were added to the upper compartment of the trans-well inserts and exposed to different concentrations of F16 (0.1-20 μM) and TMZ (10-400 μM). The lower chamber was filled with 600 μL of EMEM medium supplemented with 10% fetal bovine serum. Then, trans-well plates were incubated at 37° C. under 5% CO₂ for 24 h to allow for the migration of U87MG cells across the porous membrane. The non-migrating cells on the top chamber were removed gently with a cotton swab. The migrated cells at the bottom of the chamber were fixed in 70% ethanol and stained with crystal violet at room temperature for 20 min. Then, the trans-well inserts were rinsed with distilled water, until excess dye was removed, and then the trans-well inserts were allowed to dry. Five different fields per well were captured with a Leica microscope (DMI 3000 B; IL, USA) using 10× magnification, and the number of cells that penetrated the membrane was counted using ImageJ software (NIH Image, Bethesda, Md., USA).

### Invasion Assay

The above-described Cell Migration Assay measures the number of cells traversing a porous membrane, while the Cell Invasion Assays monitor cell movement through extracellular matrix such as Matrigel®. The U87MG cell invasion assay was performed using Corning® BioCoat™ Matrigel® Invasion Chamber that was pre-coated with BD Matrigel matrix (Corning, N.Y., USA). The 8 μm pores of the 24-well membrane inserts allow the single cells to invade. After rehydration of the Matrigel with growth medium, 5×10⁴ cells suspended in 500 μL basal medium without FBS were added to the upper chamber of the Corning® BioCoat™ Matrigel® inserts and exposed to different concentrations of F16 (0.1-20 μM) and TMZ (10-400 μM). The lower chamber was filled with 750 μL of EMEM medium supplemented with 10% fetal bovine serum. Then, the assay plates were incubated at 37° C. under 5% CO₂ for 24 h to allow for invasion of U87MG cells across the porous membrane. The non-invading cells remaining on the top chamber were removed gently with a cotton swab. The invaded cells found at the bottom of the chamber were fixed in 70% ethanol and stained with crystal violet at room temperature for 20 min. Then, the inserts were rinsed with distilled water until excess dye was removed and let to dry. Five different fields per well were captured with a Leica microscope (10× magnification), and the number of cells that penetrated the membrane was counted using ImageJ software.

### Soft Agar Colony Formation Assay

The assay was carried out in 6-well plates coated with 0.6% agarose containing EMEM. Five thousand cells of U87MG suspended in EMEM containing 0.3% low melting agarose were added to the solidified 0.6% agarose of each well. Cells were treated with F16 (10 & 20 μM), TMZ (200 & 400 μM) and a combination of both (F16 20 μM+TMZ 400 μM). After two weeks, the cells were washed with PBS, fixed in methanol for 15 min, and stained with 0.005% crystal violet for 15 min. Five different fields per well were captured with a Leica microscope (2.5× magnification), and the number of colonies counted. Three independent experiments were carried out for each assay.

### Reverse Transcription Polymerase Chain Reaction (RT-PCR) Analysis

For the RT-PCR analysis, total RNA was extracted from the treated and non-treated U87MG cells using RNeasy Kit according to the manufacturer's instructions (Qiagen, Valencia, Calif., USA). The RT-PCR reaction mixture (50 μL) consists of 1×AMV/Tfl, 1 mM MgSO₄, 0.2 mM dNTPs, 1 μM each of forward and reverse primers (list in Table 1) and 0.1 u/μL of each Tfl DNA polymerase and AMV Reverse Transcriptase. The RT-PCR products obtained from this reaction were electrophoresed on 1.5% agarose gels containing non-mutagenic fluorescent DNA dye (VWR Life sciences, Radnor, Pa., USA). The cDNA bands were visualized and captured using Bioimaging system (UVP, Upland, Calif., USA). RT-PCR products were compared by measuring the band intensity using ImageJ software.

### Western Blot Analysis

Proteins from both cell lysates and cell supernatants were used to conduct western blotting. After 24 h of treatment, U87MG cells extracted from both the control and treated groups by using RIPA (Radio immunoprecipitation assay) lysis buffer containing protease inhibitor cocktail (Santa Cruz Biotechnology, Inc. Dallas, Tex., USA). For supernatant collection, the cell culture media were separated and centrifuged at 5000 rpm for 5 min at 4° C. to remove the cell debris. After centrifugation the cell culture media were concentrated using Amicon Ultra-15® centrifugal filter, with a molecular weight cut-off limit of 10 kDa, at 4,000 rpm for 15 min at 4° C. Total protein content was determined using bicinchoninic acid (BCA) assay method (ThermoFisher Scientific, Rockford, Ill., USA). For protein separation 5-12% of sodium dodecyl sulfate-polyacrylamide gel electrophoresis (SDS-PAGE) was prepared as described by Laemmli [26]. Equal amounts of protein samples were loaded and subjected to electrophoresis, and then transferred to the nitrocellulose membrane (GE Healthcare BioSciences, Pittsburgh, Pa., USA). After blocking with 5% non-fat dry milk solution, the membranes were probed with suitable VEGFR-2, p-VEGFR-2 (Tyr 1175), AKT, p-AKT (Ser473), ERK1/2, p-ERK1/2, p53, p21, Bax, Bcl-2, MMP-2 and MMP-9 primary antibodies (1:1000 dilution). Membranes were subsequently incubated with a secondary antibody that was conjugated to horseradish peroxidase (HRP) enzyme and developed using the LumiGLO, chemiluminescence, substrate system (KPL biosolutions, USA). As a loading control, β-actin western blot was used in the analysis. The protein band intensity was quantified using ImageJ software.

### Animal Model

The glioblastoma xenograft model was developed using 8-10 weeks old male athymic nude (Nu/Nu) mice weighing approximately 25 g (Charles Rivers, US). All animals were housed in pathogen-free ventilated cages under environmentally controlled conditions of humidity and temperature (22° C.; 12:12 h light-dark cycle) with free access to pathogen-free food and water. All animal care and experiments were performed in accordance with the guidelines and approval of the Institutional Animal Care and Use Committee (IACUC) of Nova Southeastern University (NSU), Ft. Lauderdale, Fla. Animals were subcutaneously injected into the right flank of each mouse, with 4×10⁶ of U87MG glioblastoma cancer cells suspended in 100 μL of PBS mixed with Matrigel (BD Biosciences). Three weeks later, once the mice developed well-palpable tumors, they were divided randomly into four groups: group I was the untreated control, group II was treated with F16 (100 mg/kg), group III was treated with Temozolomide (50 mg/kg) and group IV was treated with F16 (100 mg/kg) and 3 h later Temozolomide (50 mg/kg). The experimental mice were treated once in every 2 days for the period of 16 days. At the end of the treatment, the tumors were isolated and then the tumor length (L) and width (W) were measured to calculate the tumor volume (TV) according to the formula: TV=1/2×(L×W²). To determine the tumor inhibitory effects of F16 and TMZ treatments, the inhibition ratio (IR) was calculated using the formula:

$$I R ( \% ) = [ 1 - ( \sum \frac{T V in treatment group}{T V in control group} ) \times 100.$$

At the end of the treatment, all the animals in the control and experimental groups were sacrificed and tumors were excised and weighed.

### Statistical Analysis

The data presented herein represent mean±SD values from at least three independent experiments. Statistical analyses were performed using a one-way analysis of variance and the differences between means were tested by Tukey's multiple comparison test. The value of p<0.05 was considered as statistically significant. Prism GraphPad (Mac OS X version 7.0b) was used to generate graphs and perform statistical analysis.

### Results

### Effect of F16 and TMZ on U87MG Cell Viability

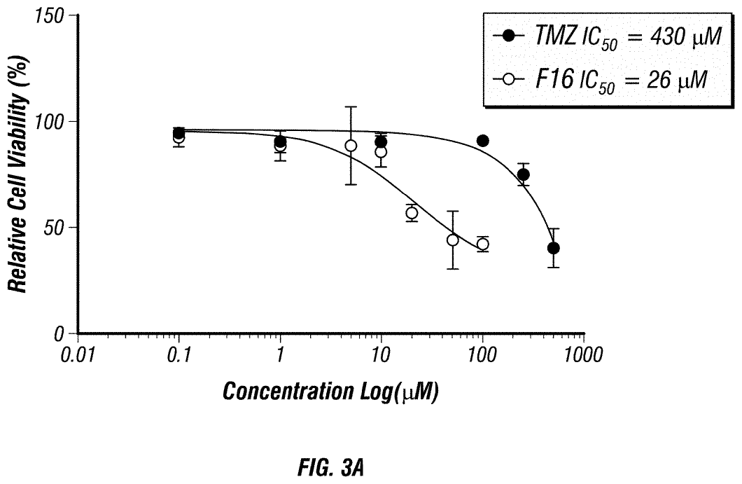

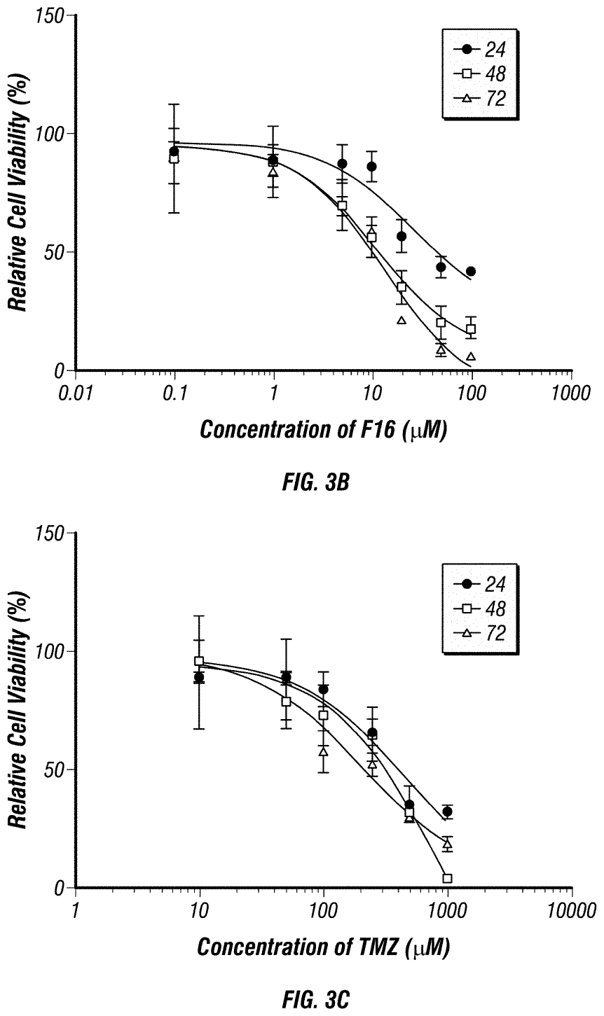

The inhibitory effects of F16 on the proliferation of U87MG cells using MTT assay and TBDE were confirmed. The percentage of viable cells obtained in the MTT assay after 24 h of treatment with varying concentrations of F16 (0.1-100 μM) and TMZ (0.1-500 μM) was shown in FIG. 3A. The proliferation of U87MG cells was markedly decreased after F16 treatment in a concentration-dependent manner. After 24 h of incubation, 50% reduction of U87MG cell viability was found to be achieved in the concentration of 26±4 μM of F16 and with 430±10 μM of TMZ. In addition, TBDE method was performed to confirm MTT result. The proliferation of U87MG cells was significantly decreased after F16 treatment in a concentration- and time-dependent manner. The maximum percentage of U87MG cells death after treatment with F16 (100 μM) for 24, 48 and 72 h were 58%, 82% and 95%, respectively (FIG. 3B). The maximum percentage of U87MG cells death after treatment with TMZ (1000 μM) for 24, 48 and 72 h were 68%, 95% and 82%, respectively (FIG. 3C).

### F16 Changed Cell Morphology in Concentration-Dependent Manner

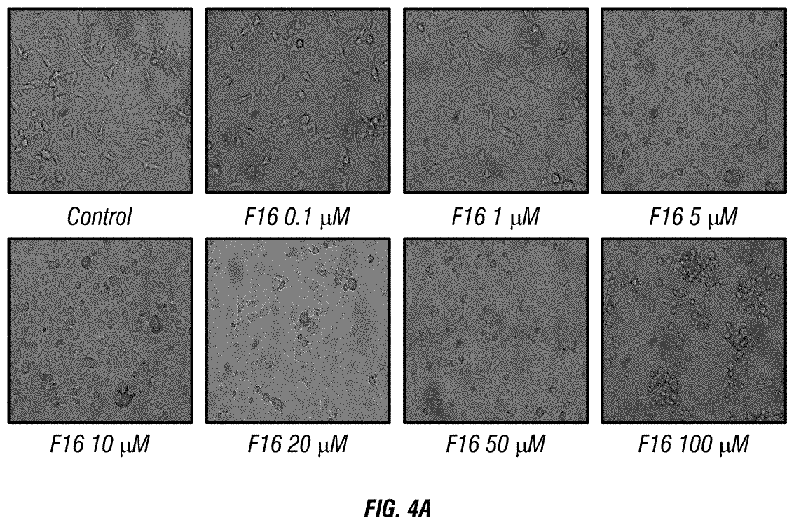

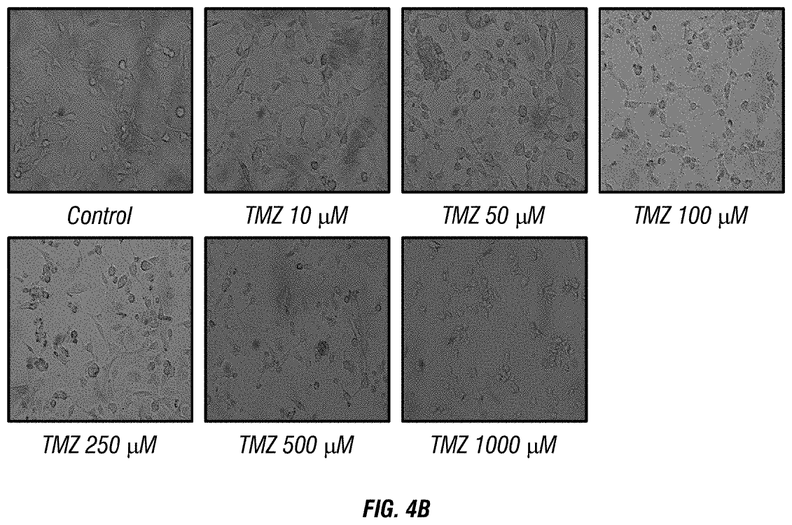

In addition to the cell death, F16 was able to induce changes in the cellular morphology of U87MG cells, preceding the cell death, in a concentration-dependent manner (FIG. 4A). Therefore, it was proposed that F16 might inhibit cell migration and invasion in U87MG cells. Considering the observation that there was no significant death of U87MG cells when treated with 10 and 20 μM of F16 for 24 h and simultaneously morphological changes were observed, these concentrations were selected for further studies. Similarly, 200 and 400 μM of TMZ were selected for further studies, which were below its IC50 value. As anticipated, both F16 and TMZ changed cellular morphology of the U87MG cells and showed concentration-dependent effects up to 100 μM and 1000 μM, respectively (FIG. 4B).

### F16 Inhibited Migration in U87MG Cells

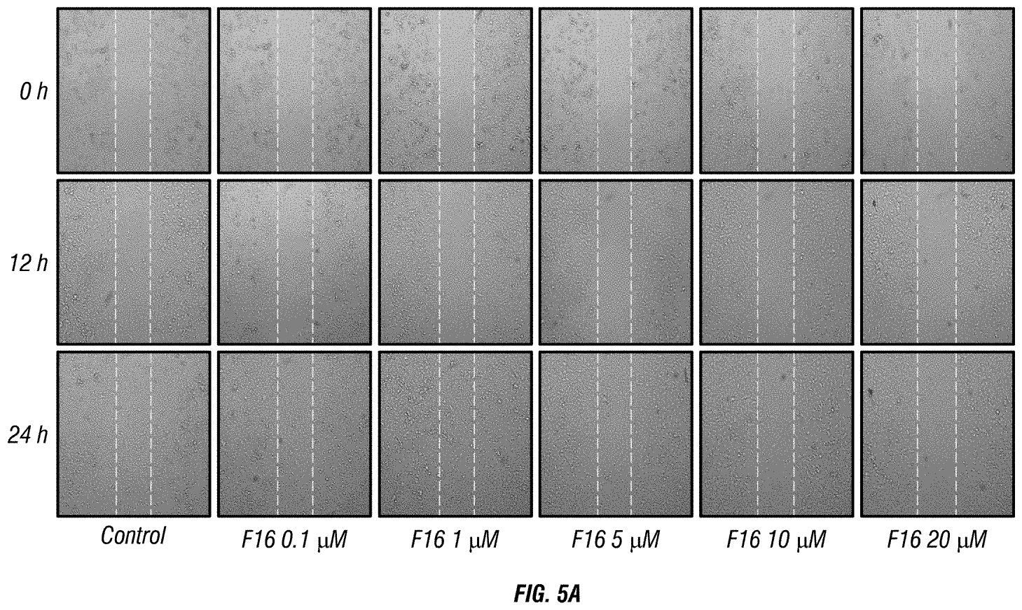

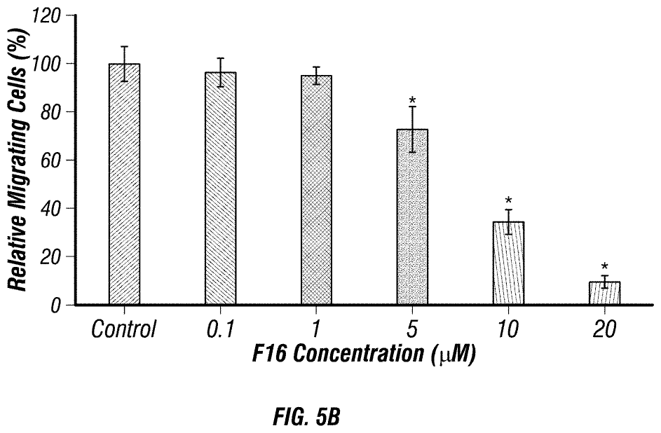

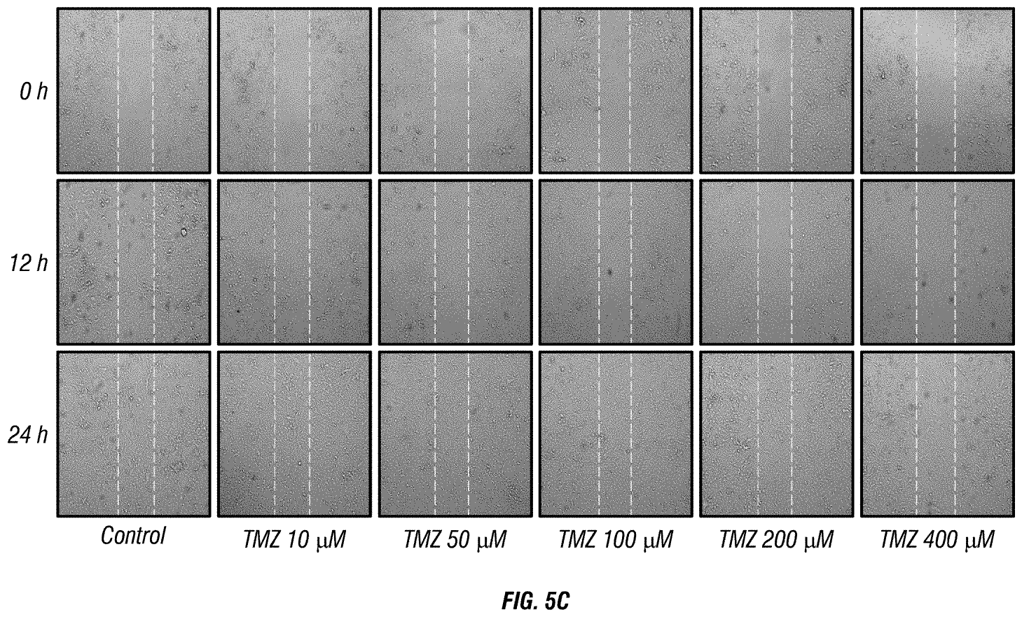

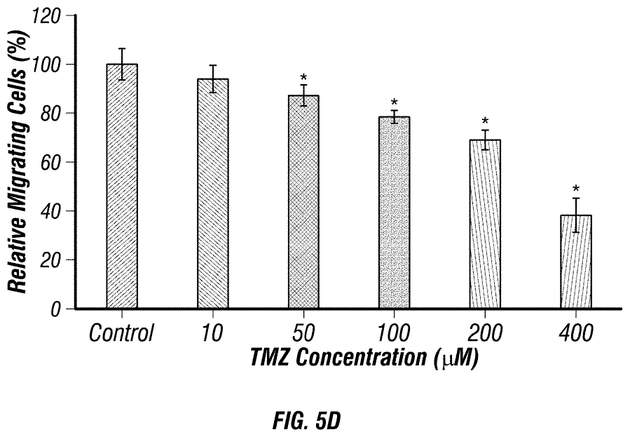

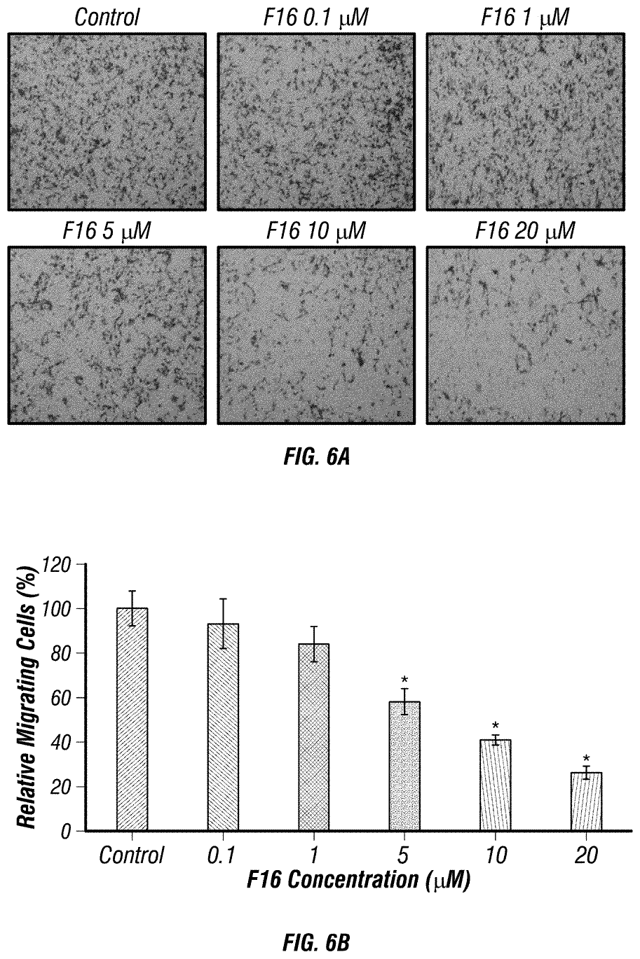

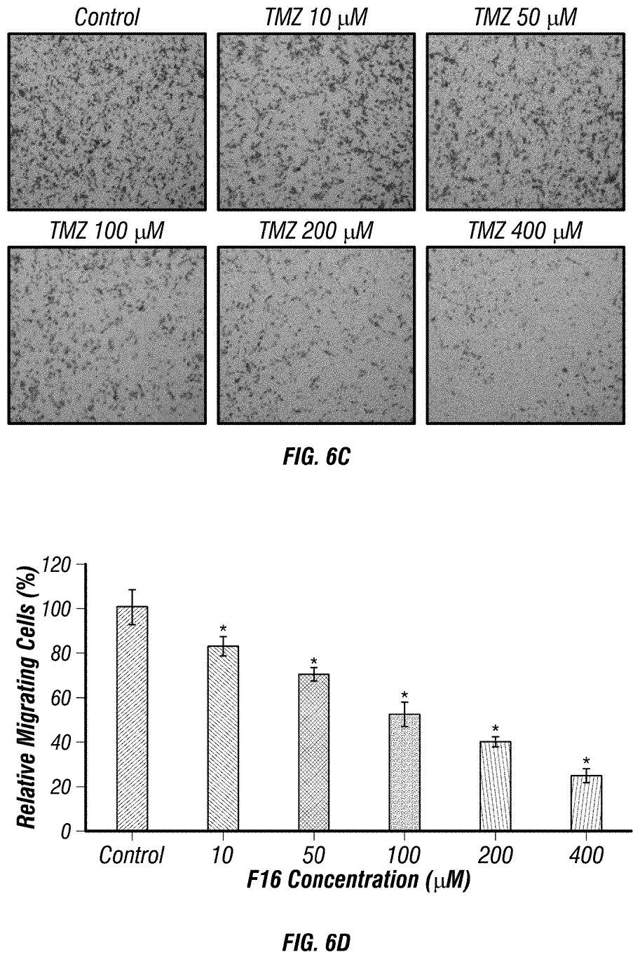

To further confirm the anti-angiogenic property and the effects of F16 on the migration of U87MG cells, the commonly used scratch assay (wound healing assay) was performed. The results showed that F16 was able to significantly inhibit the migration ability of U87MG cells in a concentration-dependent manner (FIGS. 5A-B). After 12 and 24 h post-scratch, no migration was observed when the cells were treated with 20 μM of F16, which indicated clearly that F16 has a strong ability to inhibit U87MG cell migration. However, cells treated with 400 μM of TMZ showed inhibition of migration up to 12 h post-scratch, but they started to migrate after that (FIGS. 5C-D). Similarly, F16 exhibited consistent inhibitory effects on cell migration as shown by the results obtained from the trans-well migration assay. About 80% of U87MG cells, treated with 20 μM of F16 for 24 h, were trapped in the upper compartment as compared to untreated cells indicating potent anti-migration effects of F16 (FIGS. 6A-B). Consequently, about 80% of U87MG cells were trapped in the upper compartment when treated with 400 μM of TMZ as compared to untreated cells (FIGS. 6C-D).

### F16 Inhibited Invasion in U87MG Cells

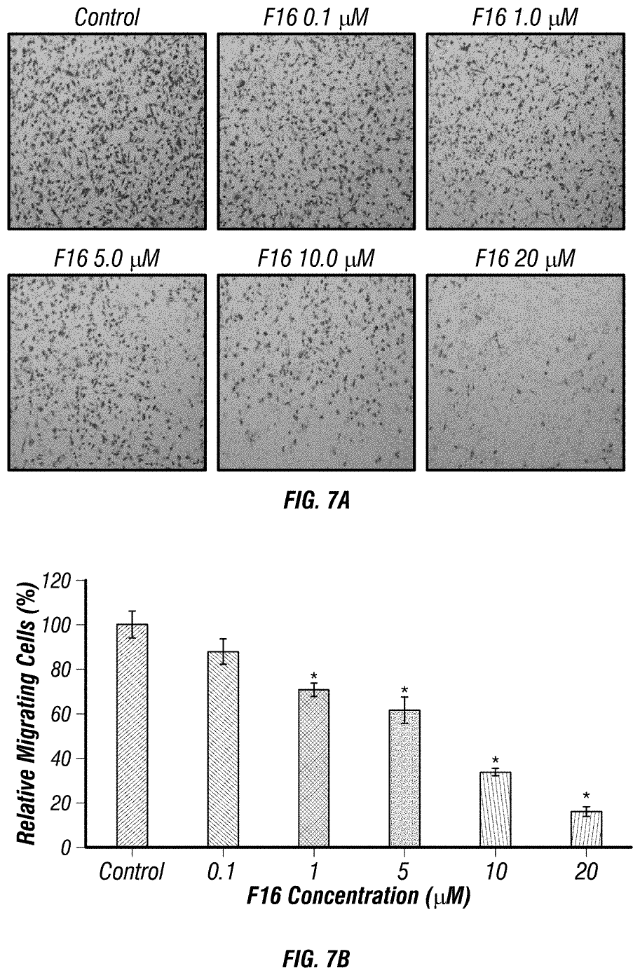

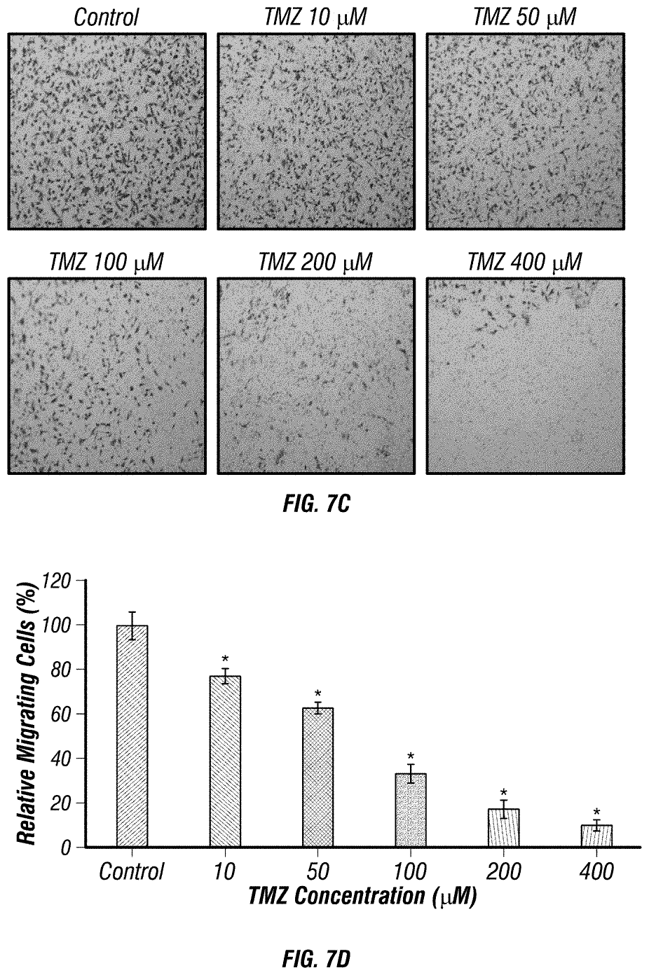

To determine whether F16 weakens the cell invasive potential, Matrigel® invasion assays were conducted using the trans-well plates. The results showed that U87MG cells invading through the Matrigel® matrix was significantly decreased in a concentration-dependent manner after 24 h of treatment with F16 versus untreated control cells (FIGS. 7A-B). As shown in FIGS. 7A-B, F16 diminished the cell invasive ability significantly. However, with much higher concentrations (400 μM), similar results were obtained with TMZ treatment, which confirmed that TMZ could marginally inhibit the invading ability of U87MG cells in a concentration-dependent manner (FIGS. 7C-D).

### F16 Decreased Anchorage-Independent Growth in U87MG Cells

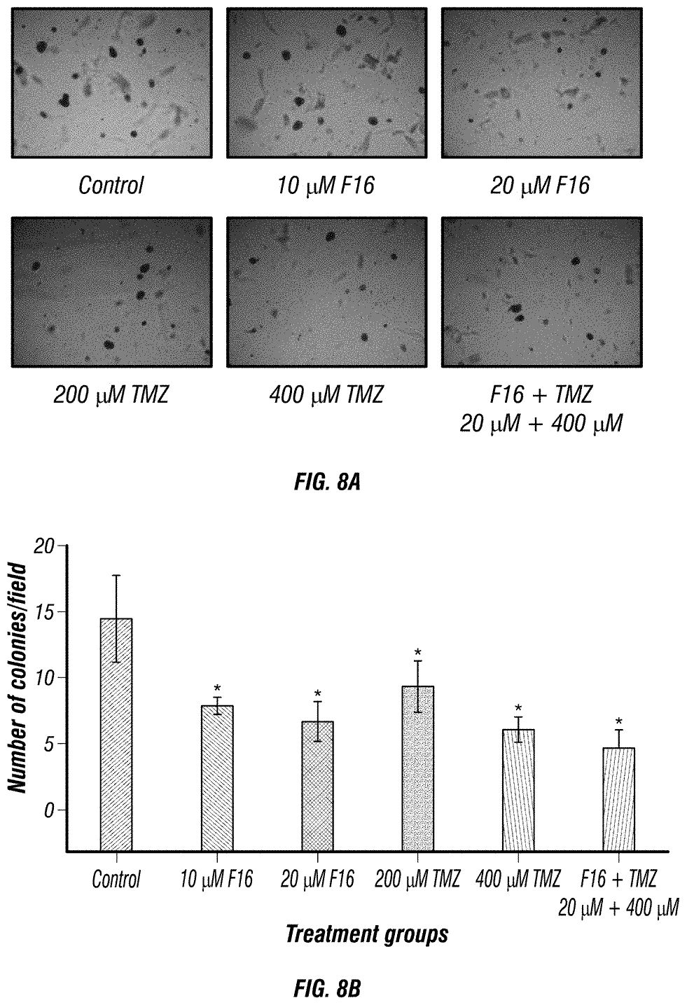

To explore the effect of F16 on anchorage-independent growth of U87MG cells, a soft agar colony formation assay was performed. The results showed that the number of anchorage-independent colonies was significantly reduced after treatment with F16 compared to untreated control cells (FIGS. 8A-B). Also, similar results were obtained with TMZ, and a combination (F16+TMZ) treatment (FIGS. 8A-B). However, there is no significant reduction in the combination (F16 20 μM+TMZ 400 μM) compared to F16 (20 μM) and to TMZ (400 μM).

### Determination of Gene Expression in U87MG Cells Using RT-PCR

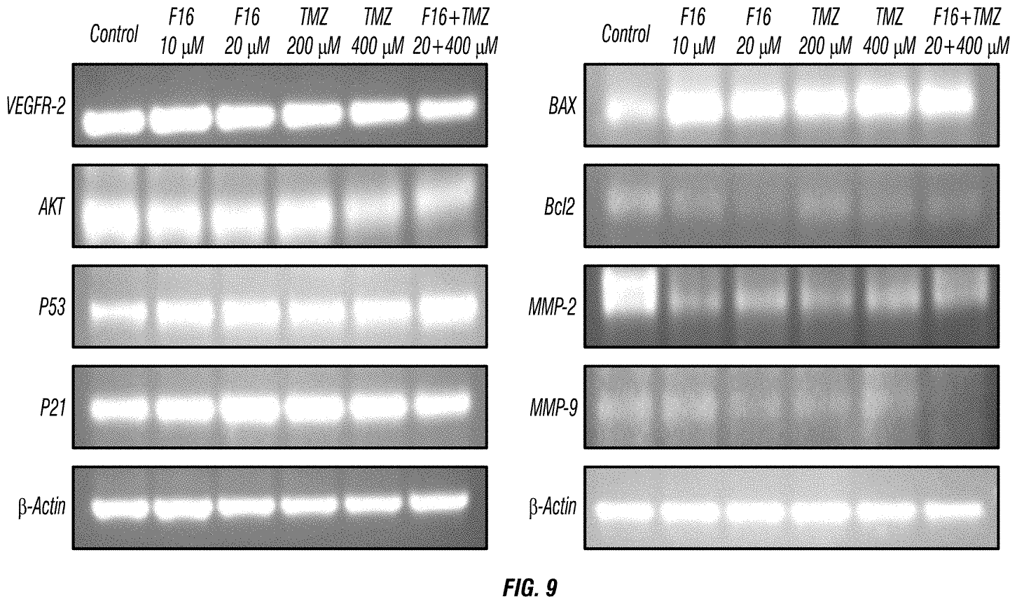

In order to further strengthen the findings, FIG. 9 shows the expression levels of selected genes in U87MG treated and untreated cells. The difference in band intensities obtained through RT-PCR indicates the differences in mRNA levels of the corresponding genes. The VEGFR-2 and AKT mRNA levels were down-regulated in the TMZ (400 μM) and F16+TMZ combination (20 & 400 μM) compared to the control. Interestingly, p53 and Bax mRNA levels were significantly up-regulated in F16 (10 & 20 μM) treated cells along with TMZ (200 & 400 μM) and F16+TMZ combination treated cells. Moreover, slight elevation in the mRNA level of p21 was observed in the F16 and TMZ treated cells compared to the control. Notably, mRNA levels of Bcl2, MMP-2 and MMP-9 were markedly down-regulated in the individual treatments with F16, TMZ, and also in the combination treatment.

### Inhibition of VEGFR-2 Phosphorylation and Downstream Signaling

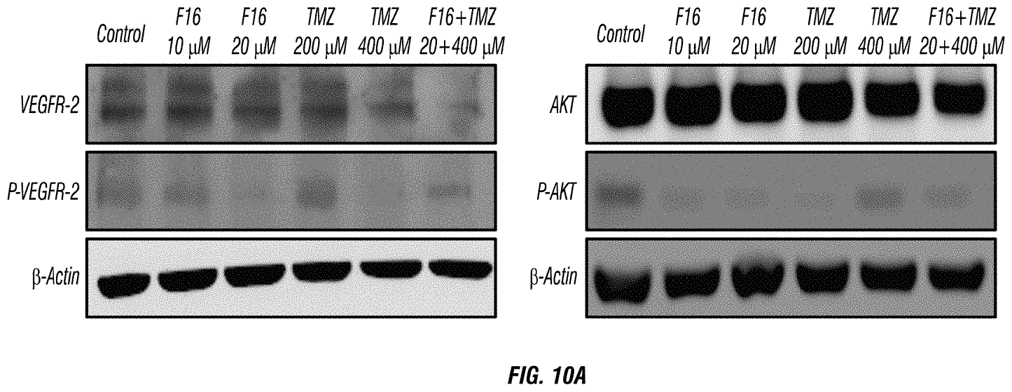

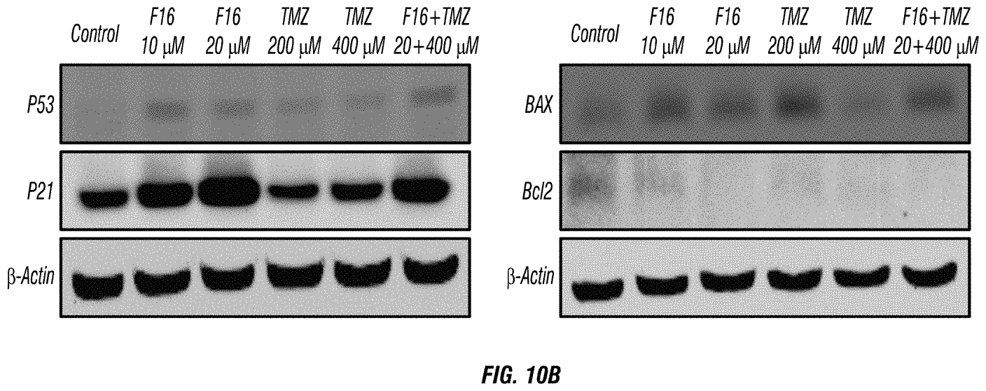

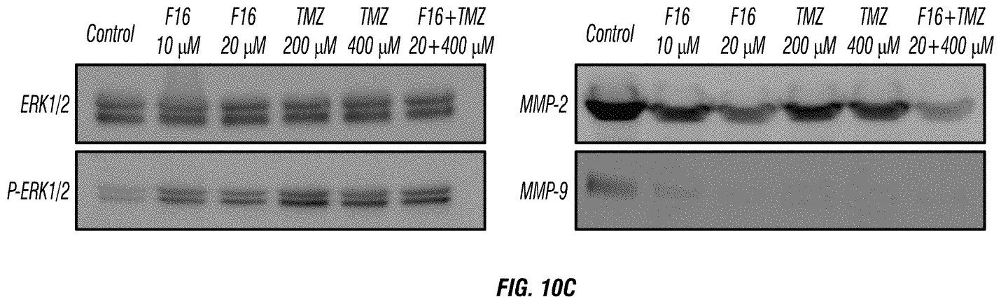

Previous studies have clearly indicated that prevention of VEGFR-2 activity could significantly limit the angiogenesis process which plays a critical role in tumor progression [27]. The level of phospho-VEGFR-2 (Tyr 1175), which is the active form of VEGFR-2, was significantly decreased after F16 treatment (FIG. 10A). Moreover, p-AKT expression at Ser473 site, a key molecular downstream target of VEGFR-2, was also significantly inhibited by F16 in the U87MG cells (FIG. 10A). These results indicated that F16 had the ability to attenuate AKT-dependent cell survival. Also, similar results were obtained with TMZ and combination (F16+TMZ) treatments.

### F16 Induced Cell Cycle Arrest and Apoptosis

To better understand the role of F16 in cell cycle arrest and apoptosis, expression of proteins p53, p21, Bax and Bcl2 was analyzed. The expression of p53, a well-established tumor suppressor gene, was upregulated after F16 treatment and combination treatment, but showed lesser increase in the expression levels after treatment with TMZ alone (FIG. 10B). In addition, the p21 expression was significantly upregulated after F16 and combination treatments (FIG. 10B). Surprisingly, the expression of p21 was markedly downregulated with TMZ treatment (FIG. 10B). Moreover, the Bax expression was also increased after F16, TMZ and combination treatments, while the expression of Bcl2 was inhibited with the same treatments (FIG. 10B). These findings suggest that p53 overexpression induces cell cycle arrest and apoptosis through p21 and Bax dependent pathways in U87MG cells.

### Effects of F16 on ERK1/2, MMP-2, MMP-9 and Cell Invasion

ERK1/2 is an important subfamily of mitogen-activated protein kinases that controls a broad range of cellular activities and physiological processes. The expression of p-ERK1/2 was upregulated after F16, TMZ and combination treatments (FIG. 10C). Furthermore, MMP-2 and MMP-9 expressions were downregulated after F16 treatment (FIG. 10C). These results showed the ability of F16 to activate ERK1/2 in a sustained way which appears to contribute to the downregulated expression of MMP-2 that was resulting in the inhibition of cell invasion. Interestingly, similar results were obtained with TMZ and combination treatments.

### Inhibition of U87MG Derived Xenograft Tumor Growth by F16

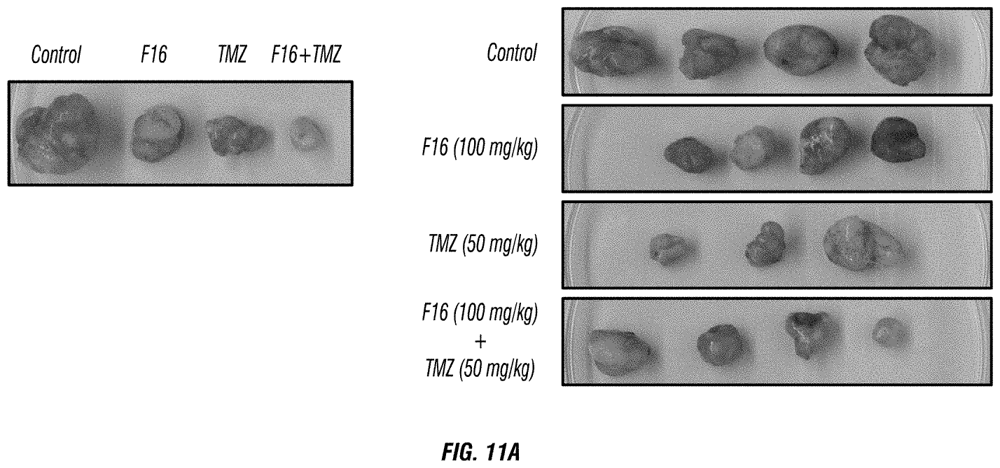

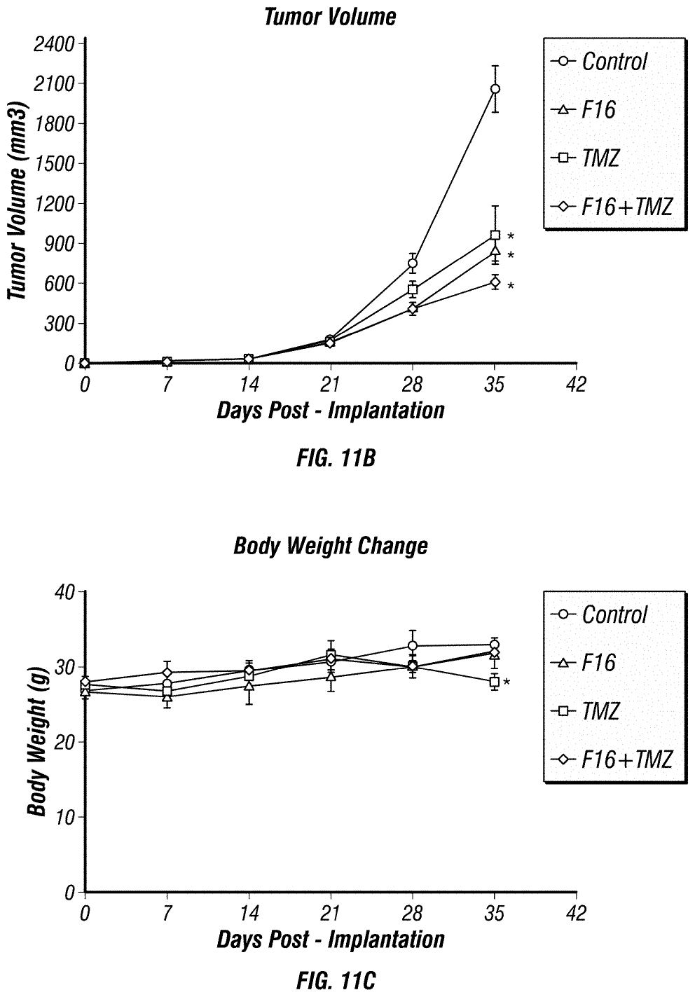

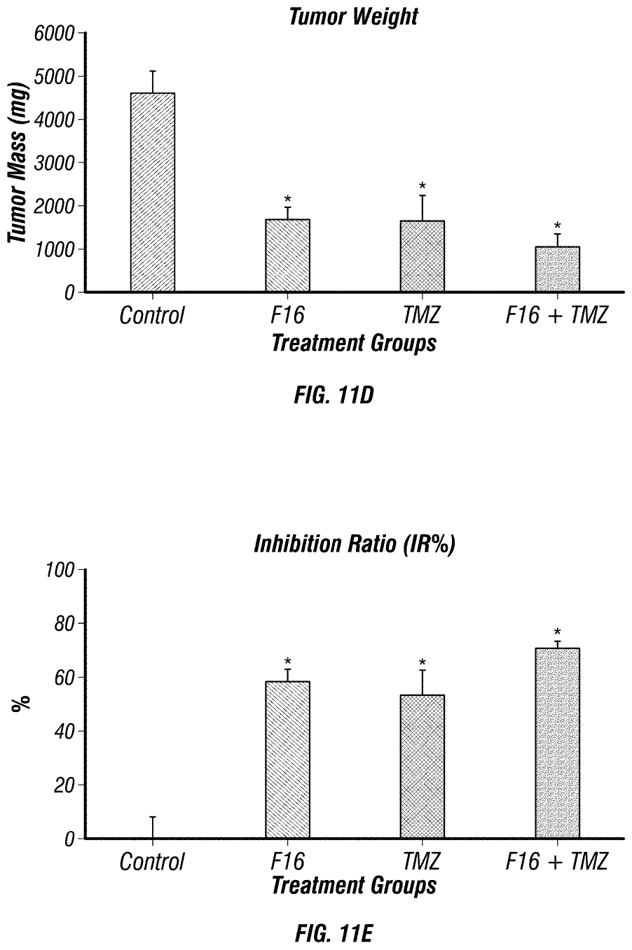

To further investigate the in vivo tumor growth inhibitory effects of F16, a subcutaneous glioblastoma xenograft model using U87MG cells was established as described earlier in the materials and methods section. Previous studies have indicated that U87MG xenograft model is considered to be one of the most widely utilized experimental models available for pre-clinical testing of glioblastoma [28, 29]. Therefore, once the tumor was fully established mice were randomized into four groups, as described before and treated intraperitoneally with F16, TMZ, and F16+TMZ combination for 16 days. Representative pictures of excised tumors are shown in FIG. 11A. The results clearly showed that mice implanted with U87MG tumors showed 58%, 53% and 70% suppression of tumor growth after treatment with F16 (100 mg/kg), TMZ (50 mg/kg) and F16 (100 mg/kg)+TMZ (50 mg/kg), respectively, for 16 days (FIG. 11B). Interestingly, the tumor growth inhibitory effect of F16 monotherapy was comparable to TMZ at the indicated dose with no signs of toxicity in F16 group. However, the combination of F16 with TMZ, a standard care of treatment for glioblastoma cancer, did not yield any significant reduction in tumor volume (70%) compare to the monotherapy of either F16 (58%) or TMZ (53%).

Changes in body weight of the experimental mice were also examined during the treatment period (FIG. 11C). Consistent with previous experiments, F16 treatment was well tolerated at the dose that was used in the treatment (100 mg/kg). However, symptoms of toxicity, such as weight loss, general weakness, accumulation of ascites were observed after one week of treatment in the TMZ group as well as in the combination group with the loss of one of the animals in the TMZ group. At the end of the treatment period, the tumors were excised for comparison. As shown in FIG. 11D, tumor weight was significantly lower in F16 and TMZ and combination treated groups compared to the control group. The IR % was calculated as described in the Methods section and shown in FIG. 11E.

### Discussion

The prognosis of Glioblastoma multiforme (GBM) remains poor, and the available treatment options currently provide only modest benefits with a barely significant increase in patient survival. The current standard of care for newly diagnosed patients with GBM is surgical resection followed by a course of radiation plus cytotoxic therapy with chemotherapeutic agent such as Temozolomide (TMZ) [30]. The addition of TMZ to radiotherapy increases the overall median survival by 2.6 months (total of 14.6 months) compared to 12 months of median survival for radiotherapy alone [31]. However, TMZ administration was clinically associated with severe toxicities such as genotoxicity, bone marrow suppression, teratogenicity, and severe intestinal damage [32]. Earlier studies have reported that, similar to several other cytotoxic chemotherapeutic agents in general, TMZ possess cytotoxic effects on normal cells, which are often associated the onset of secondary cancers [33]. All these shortfalls associated with TMZ have prompted scientists to develop more effective therapeutic options for the treatment of GBM. Moreover, high expression of VEGF found in GBM is also associated with poor prognosis, which provides a logical rationale to evaluate angiogenesis inhibitors as preferred drugs to treat GBM. In this context, F16, a novel small molecule that competitively blocks VEGF binding to its receptors and blocks ligand induced phosphorylation of VEGFR-2 (Tyr1175) in HUVEC and exhibits in vitro anti-angiogenic activity, was found by the instant inventors. The above-mentioned VEGFR-2 specific binding agent was shown to inhibit endothelial cell proliferation, migration, and tube formation [24].

Initially, VEGFR-2 was thought to be exclusively expressed at high levels only in endothelial cells. However, several studies conducted in the last few years have demonstrated that certain cancer cells, such as glioblastoma cells, also express the VEGFR-2 in relatively high levels [34]. Interestingly, the U87MG cell line is one of the glioblastoma cell lines that expresses high levels of VEGFR-2 [34] with high sensitivity towards TMZ treatment [35]. Because of that, the U87MG cell line was chosen as a model representing glioblastoma to test and compare the efficacy of F16 with the standard TMZ. The initial experiments were directed towards comparing the anti-proliferative effects of F16 and TMZ against U87MG glioblastoma cancer cells using MTT and TBDE assays. In the in vitro experiments, F16 exhibited higher potency against U87MG cells with an IC₅₀ of 26 μM which was 15 folds lower than IC₅₀ value (FIG. 3A) of TMZ (430 μM). The data is in agreement with the IC₅₀ values of TMZ (172-700 μM) that are reported in the literature [36-38]. Furthermore, concentration and time dependent effects of F16 in inducing cytotoxicity in U87MG using TBDE method also confirmed the IC₅₀ determination that was achieved with MTT assay. Besides that, the effects of F16 and TMZ on the anchorage-independent growth of U87MG cells (the ability of cells to grow independently on a solid surface) was tested using a soft agar colony formation assay [39]. The in vitro colony formation of U87MG cells in soft agar was significantly suppressed by F16 and TMZ compared to the control (FIGS. 8A-B), which confirmed the ability of F16 in inhibiting the anchorage-independent growth of U87MG cells.

To study the underlying molecular mechanisms that mediate F16 induced cytotoxicity in U87MG cells, phosphorylation of VEGFR-2 after F16 treatment was studied. VEGFR-2 has seven phosphorylation sites, including Tyr1175, which regulates cell proliferation and migration [40]. The results showed a significant inhibition in the level of p-VEGFR-2 (Tyr1175) in U87MG cells after F16 treatment (FIG. 10A). Recent studies have also confirmed the antagonistic action of F16 through competitive binding with VEGFR-2 [24].

As a consequence of the blockade in VEGFR-2 phosphorylation by the F16, the PI3K-AKT pathway was explored, one of the downstream targets of VEGFR-2 that plays an important role in promoting cell survival and cell cycle progression [40, 41]. Previous studies have shown that activation of AKT is involved in inhibiting apoptosis by hindering transcription factors that promote expression of pro-apoptotic genes, and enhancing transcription of anti-apoptotic genes [41, 42]. Furthermore, AKT was shown to suppress p53 mediated apoptosis in indirect manner by phosphorylation of murine double minute 2 (MDM2), which is a negative regulator of p53 [43]. On the other hand, inhibition of AKT phosphorylation was shown to promote cancer cell death and apoptosis through p53 mediated pathway [41, 43]. Thus, the results suggested that F16 could promote cell death through inhibition of AKT phosphorylation at Ser473 site and activating of p53 pathway, to eventually induce cell cycle arrest and apoptosis by up-regulation of p21 and Bax. As anticipated, F16 was able to induce expression of p53, p21, Bax and decrease expression of Bcl2 following 24 h treatment (FIG. 10B). These results clearly indicated that F16 is capable of inhibiting U87MG cells survival mediated by AKT and induces apoptosis through activation of p53 pathway.

A distinctive pathological feature of GBM cells is their ability to extensively invade surroundings containing normal brain tissues [44]. GBM cell invasion is a complex multistep process that typically starts with the degradation of extracellular matrix (ECM) by MMPs, which allows cancer cells to migrate out of the primary tumor to form secondary metastases [44, 45]. Many studies have reported that MMP-2 along with MMP-9 are highly expressed in various human glioblastoma cell lines including U87MG [46-48]. Both MMP-2 and MMP-9 degrade type IV collagen, which is the most abundant component of the basement membrane.

Therefore, degradation of collagen is a crucial step for the initiation of metastatic progression of most cancers [46]. Thus, downregulation of MMP-2 and MMP-9 expression is closely associated with inhibition of GBM cell migration and invasion [48]. The results with U87MG cells clearly showed that F16 significantly inhibited both migration and invasion at concentrations that are below the IC₅₀ values (FIGS. 5A-B, 6A-B, and 7A-B). While blocking migration and invasion, F16 treatment downregulated expression of MMP-2 and MMP-9 also (FIG. 10C). Furthermore, several studies which have reported a sustained activation of ERK1/2 signaling inhibits tumor cell invasion in many human glioblastoma cancer cells, including U87MG cells [49] and human prostate cancer cells [50]. The ERK1/2 enzyme is an important subfamily of mitogen-activated protein kinases that have been shown to have substantial roles in regulating cell proliferation, apoptosis and invasion depending on the cell types and mode of activation [51-53]. It has been shown that transient activation of ERK1/2 (<15 min stimulation) could induce proliferation, migration and invasion of cancer cells. On the other hand, opposite effects were observed with sustained activation (>15 min stimulation) of ERK1/2 [53-55] which appears to be in agreement with the results that were obtained after treating U87MG cells with F16 and TMZ (FIG. 10C).

To support the in vitro results, the efficacy of F16 in delaying glioblastoma progression using in vivo model was examined. The subcutaneous glioblastoma xenograft model (using the athymic nude mice and treat them with F16, TMZ and combinations) was successfully established. The in vivo results show that F16 significantly inhibited xenograft tumor growth suggesting that VEGFR-2 blockade using F16 treatment is efficacious in delaying glioblastoma cancer growth (FIG. 11B). Unlike mice treated with TMZ alone, F16 treatment up to 16 days showed no signs of toxicity, which is consistent with the previous studies that were conducted on different cancer models in the inventors' laboratory [24, 25]. Unpredictably, mice treated with the combination of F16 with TMZ showed no significant difference in the reduction of tumor volume compare to the mice treated with the monotherapies (FIG. 11B). Moreover, signs of increasing toxicity and intolerability were observed in the combination group. Such toxicity might be reduced if less TMZ dose was used or the interval between the administrations of the two drugs are increased.

In conclusion to Example 1, the in vitro and in vivo results clearly demonstrate high potency of F16 treatment in inhibiting U87MG cells survival, migration, and invasion. In comparison to TMZ, F16 has a potent cytotoxicity against U87MG cells with an IC₅₀ 26 μM (FIG. 3A) and has a better tolerability in mice. F16 also exhibited strong anticancer effect by delaying the tumor growth in xenograft implanted athymic nude mice.

### Example 2: Intracranial Model of Glioblastoma

Though promising results with F16 were obtained, Example 1 used a single cell line in a subcutaneous xenograft model that was responsive to the drug treatment. Therefore, utilization of another in vivo model such as intracranial brain tumor xenograft will provide further validation for its therapeutic effects towards GBM. Hence, the main focus of Example 2 was to determine the efficacy of F16 in delaying glioblastoma progression using intracranial GBM xenograft model, and to evaluate the tolerability of F16 in KP formulation to establish its safety profile using a mouse model.

Cancer remains the second leading cause of death worldwide despite great efforts and resources that are being devoted for developing newer treatment strategies and diagnostic methods [1]. Every year millions of people are diagnosed with cancer around the world, and the survival rate for those patients becomes exceptionally low mainly in the late stages. Among cancer types, glioblastoma multiforme (GBM) is one of the most aggressive and lethal types of brain cancer with a poor prognosis and only less than 5% of patients survive 5-years following diagnosis [2]. As noted above in Example 1, the current standard of care for newly diagnosed patients with GBM is surgical resection, whenever it is applicable, followed by a course of radiation plus chemotherapy such as Temozolomide (TMZ) [3]. Addition of TMZ provides a modest increase in overall survival (OS) from 12.1 to 14.6 months compared to surgical debulking followed by adjuvant radiation therapy [4, 5]. However, TMZ treatment is developed resistance and clinically is associated with severe toxicities such as genotoxicity, teratogenicity, bone marrow suppression, and severe intestinal damage [6]. Therefore, development of more effective and safer treatments for GBM is urgently needed.

One of the defining features of GBM is an abundant and aberrant vasculature [7]. Unlike normal brain vasculature, GBM vasculature is disorganized, poorly connected, tortuous, and associated with marked endothelial proliferation, resulting in regions of hypoxia [8]. Moreover, vascular endothelial growth factor (VEGF) is elevated in GBM with increased vessel permeability, vessel diameter, and abnormality in endothelial wall and basement membrane thickness [9, 10]. High expression of VEGF found in the GBM is also associated with poor prognosis, which provided a logical rationale to evaluate angiogenesis inhibitors as preferred drugs to treat GBM [11].

In preclinical and clinical studies, the use of angiogenesis inhibitors in combination with chemotherapeutic agents has shown promising results against a wide range of cancer types [12-15]. Recently, use of angiogenesis inhibitors has been emerging as a novel strategy for glioblastoma treatment due to the prominent angiogenesis that occur in GBM. So far, bevacizumab (BVZ) is the only antiangiogenic drug that has been approved by FDA for treatment of recurrent GBM. However, BVZ treatment has yielded no improvement in the overall survival (OS) and the FDA approval was based on the increase in the overall Objective Response Rate (ORR) [16, 17].

As noted above, one of the major challenges of treating brain cancers is the presence of the blood brain barrier (BBB). The BBB is a highly selective barrier and crossing this barrier is not easy for large molecules and is required small (molecular mass less than 400-500 Da) lipophilic molecules [18]. Therefore, recent interest has shifted towards exploring small molecules that can cross BBB to modulate angiogenesis and similar processes. In this context, F16, a novel small molecule (molecular weight 301.2 g/mol), has exhibited potent anti-angiogenic and anti-tumor activities via selectively antagonizing vascular endothelial growth factor receptor-2 (VEGFR-2) in both in vitro and in vivo models [19]. More importantly, the preclinical pharmacokinetics studies have shown that F16 can cross BBB and accumulate into brain regions [20]. Therefore, in Example 1, the direct effects of F16 for inhibiting the growth, angiogenesis and the migratory abilities of the U87MG glioblastoma cells (which are known to express high levels of VEGFR) were tested. The in vitro studies confirmed potent inhibitory effects of F16 towards the migration and invasion of U87MG cells and revealed potent cytotoxic effects (IC₅₀ 26 μM) against U87MG cells in comparison to Temozolomide (IC₅₀ 430 μM) treatment. In addition, F16 inhibited the phosphorylation of VEGFR-2 through competitive binding and induced cell cycle arrest and apoptosis by activating p53 pathway in U87MG cells. Furthermore, the in vivo results with ectopically implanted xenograft model confirm the fact that F16 can significantly inhibit tumor growth in the mice implanted with U87MG glioblastoma cell line.

Example 2 utilizes another in vivo model, such as intracranial brain tumor xenograft, to provide further validation for F16 therapeutic effects towards GBM. Hence, a main focus of Example 2 was to determine the efficacy of F16 in delaying glioblastoma progression using intracranial GBM xenograft model, and to evaluate the tolerability of F16 in KP formulation to establish its safety profile using a mouse model.

### Material and Methods

### Cell Line and Reagents

U87MG, a human glioblastoma cell line, was purchased from ATCC (Manassas, Va., USA) and maintained in Eagle's minimum essential medium (EMEM) supplemented with 10% fetal bovine serum, 2 mM L-glutamine, 1.5 g/L sodium bicarbonate and 1% penicillin/streptomycin. Cells were incubated at 37° C. with 95% air and 5% CO₂ in a humidified incubator. U87MG cells were used in assays, when the cell passages were between 3 and 9. The F16 and TMZ (Sigma-Aldrich, St. Louis, Mo., USA) were prepared as a solution in dimethyl sulfoxide (DMSO). All other chemicals used in these experiments were of research grade.

U87MG Cells Luciferase Gene Transfection (pcDNA3.1-Luc)

For the purpose of developing a cell line for xenograft imaging experiments, the U87MG cell line with 90-95% confluency (6 well plates) was used for transfection with Lipofectamine 2000. On the day of transfection, cells were replenished with fresh medium without any antibiotics. For the transfection process, complex A (10 μg pcDNA3.1-Luc+15 μl of PLUS reagent in 100 μl of serum free medium) and complex B (12 μl Lipofectamine 2000 in 100 μl of serum free medium) were prepared separately and incubated for 15 min at room temperature. Complex A and B were combined and incubated for further 15 min at room temperature. This solution (200 μl) added to the plated cells containing 800 μl appropriate medium (serum and antibiotic free) and incubated for further 5 hrs in a 5% CO₂ incubator at 37° C. Furthermore, 1 mL of growth medium containing 20% serum without antibiotic was added on transfected wells and incubation was further continued for another 72 hrs (with U-87MG cells) to allow stable transfection.

### Measurement of Luciferase Signal in U87MG-Luc Cells

To measure the cultured luciferase gene transfected cells (U87MG-Luc cells), we imaged the luciferase signal with different cell numbers (1×10⁴-3×10⁵), by adding phosphate buffer saline with D-luciferin (Fisher Scientific, USA) at the concentration of 0.15 mg/ml. U87MG-Luc cells were imaged 10 minutes after incubation with D-luciferin at room temperature. The measurement of the luciferase signal was analyzed using the Bruker Xtreme II (Bruker, Billerica, Mass.).

### Animal Model

For tolerability study, 8-10 weeks old male BALB/c mice weighing approximately 25 g were used (Charles Rivers, US). For intracranial study, 8-10 weeks old female athymic nude (Nu/Nu) mice weighing approximately 25 g were used (Taconic Biosciences, US). All animals were housed in pathogen-free ventilated cages under environmentally controlled conditions of humidity and temperature (22° C.; 12:12 h light-dark cycle) with free access to pathogen-free food and water. All animal care and experiments were performed in accordance with the guidelines and approval of the Institutional Animal Care and Use Committee (IACUC) of Nova Southeastern University (NSU), Ft. Lauderdale, Fla.

### Drug Preparation

F16 (100 mg/kg) was dissolved in a 10% DMSO+90% KolliphorEL (KP). TMZ (50 mg/kg) was dissolved in 10% DMSO+90% phosphate-buffered saline (PBS). All drugs were prepared fresh before the scheduled injection [21]. The total volume of injection was 100 μL/mouse for all experiments which was administered intraperitoneally.

### Experimental Procedures

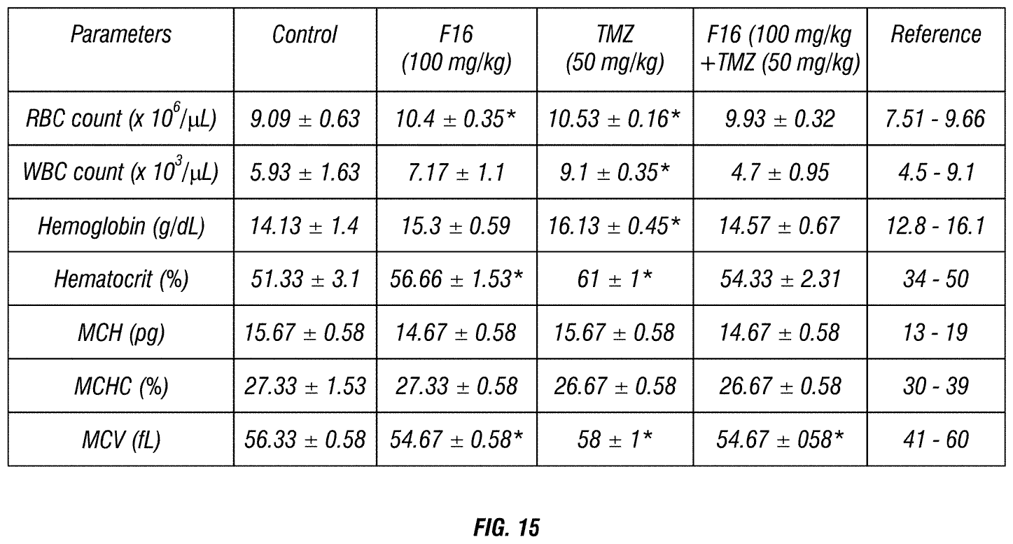

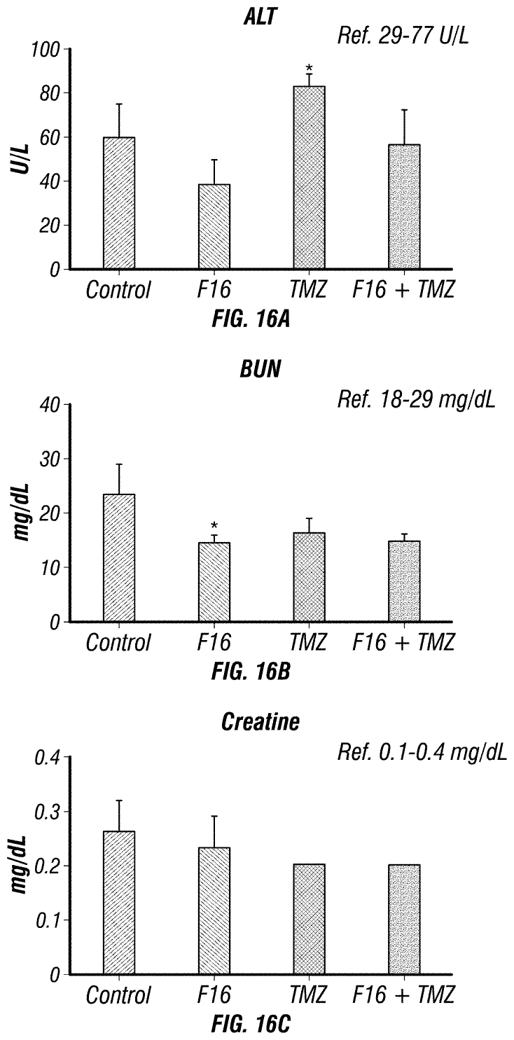

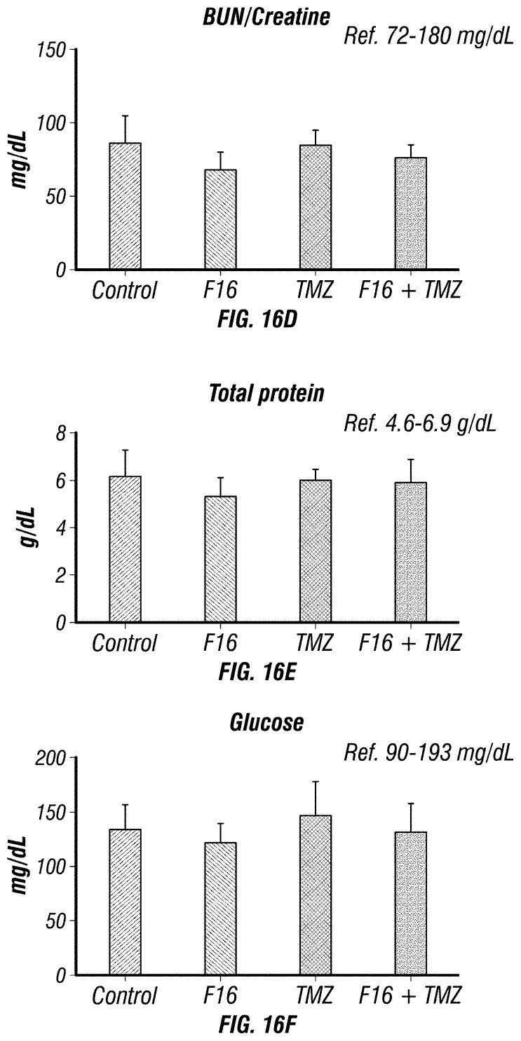

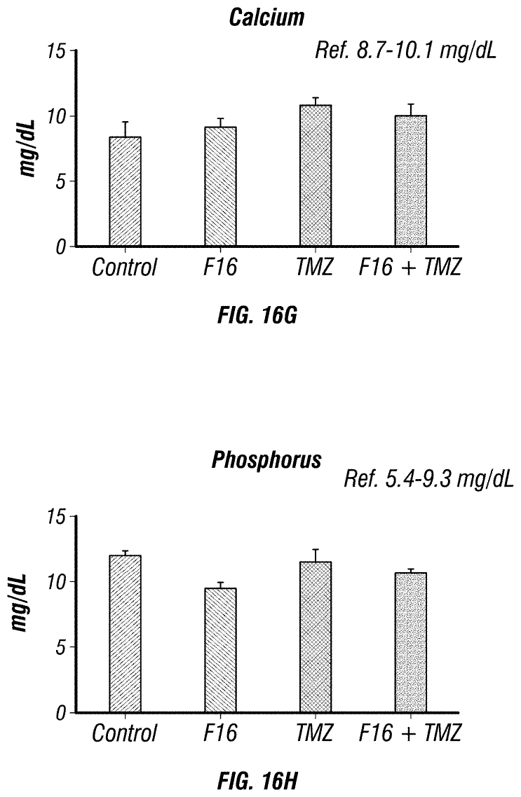

For tolerability studies, BALB/c mice were randomly assigned to 4 different treatment groups (FIG. 15: Table 1). At the end of the treatment period, blood samples from all the mice were collected and sent to the Department of Comparative Pathology at the University of Miami (City of Miami, Fla.) to analyze the hematological and biochemical parameters.

For intracranial study, the glioblastoma xenograft model was developed using athymic nude (Nu/Nu) mice. Briefly, mice were placed under general anesthesia (intraperitoneal injection of 100 mg/kg Ketamine and 10 mg/kg Xylazine) and were positioned in the stereotaxic device. A median incision of ˜1 cm was made, and a burr hole was drilled into the right striatum of the skull (1.0 mm forward and 2.0 mm lateral to the bregma). Subsequently, U87MG cells expressing the luc reporter gene (2×10⁵ cells in 3 μL PBS) was injected using a 10-μl Hamilton syringe at the rate of 1 μL/min at a depth of 3 mm. Once injection was completed, the needle was kept in place for 2 minutes and then slowly removed, and the hole was sealed with a sterile bone wax. The incision was closed, and triple antibiotic ointment was applied. One week after tumor cell transplantation, mice were divided randomly into five groups (n=5 in each group): 1) control treated with DMSO in PBS, 2) control treated with DMSO in KP, 3) treated with F16 (100 mg/kg), 4) treated with temozolomide (50 mg/kg) and 5) treated with F16 (100 mg/kg) and 3 h later treated with temozolomide (50 mg/kg). One more group with no tumor implant was added to the study as a negative control (n=5). The experimental mice were treated twice per week for 3 weeks. After the treatment was completed, mice were maintained without any treatment until they showed serious illnesses and then euthanized using the Euthanex CO₂ smart box. Brains and tumors of the euthanized mice were isolated for histology and immunohistochemistry (IHC) studies.

### Bioluminescence Imaging In Vivo

Bioluminescence imaging (BLI) was used to assess and confirm tumor growth in intracranial xenograft. BLI was carried out in vivo using the Bruker Xtreme which is a sensitive optical X-ray machine designed for preclinical in vivo study based on BLI concept. Briefly, mice were injected intraperitoneally with D-luciferin (Sigma) dissolved in saline at a dose of 150 mg/kg body weight. Immediately after the injection, mice were anesthetized by isoflurane and series of bioluminescent images was acquired with 3-minutes acquisition intervals for approximately 20 minutes, by which time, the luciferin had been washed out. The image with the peak BLI intensity was used for quantification in units of photon counts.

### Histology and Immunohistochemistry

Histological analyses to evaluate the tumor histology and the effect of the experimental drug against the tumor was performed. Surgically resected tumors in brain tissues were rinsed in 1×PBS to remove blood for the histology and IHC preparations. The specimens from each experimental group were fixed in 10% Neutral Buffered Formalin (NBF) and shipped to Molecular Pathology Core, University of Florida to process the samples for further histology and IHC preparations. The microscopic images and data were received from the facility. Samples of IHC were incubated with primary mouse monoclonal anti-CD31 antibody (1:100 dilution; Cell Signaling Tech. Inc) and the secondary antibody biotin-labeled rabbit anti-mouse IgG (1:500; Nichirei, Tokyo, Japan) was performed using a DAB staining kit. The sections were counterstained with hematoxylin. For H & E (hematoxylin and eosin) staining, samples were stained with Harris' hematoxylin solution and were followed by eosin solution in Molecular Pathology Core, University of Florida.

### Statistical Analysis

The data presented here represent mean±SD values from at least three independent experiments. Statistical analyses were performed using a one-way analysis of variance and the differences between means were tested by Tukey's multiple comparison test. The value of p<0.05 was considered as statistically significant. Prism GraphPad (Mac OS X version 7.0b) was used to generate graphs and perform statistical analysis.

### Results

### Selection and Measurement of Luciferase Signal in U87MG-Luc Cells

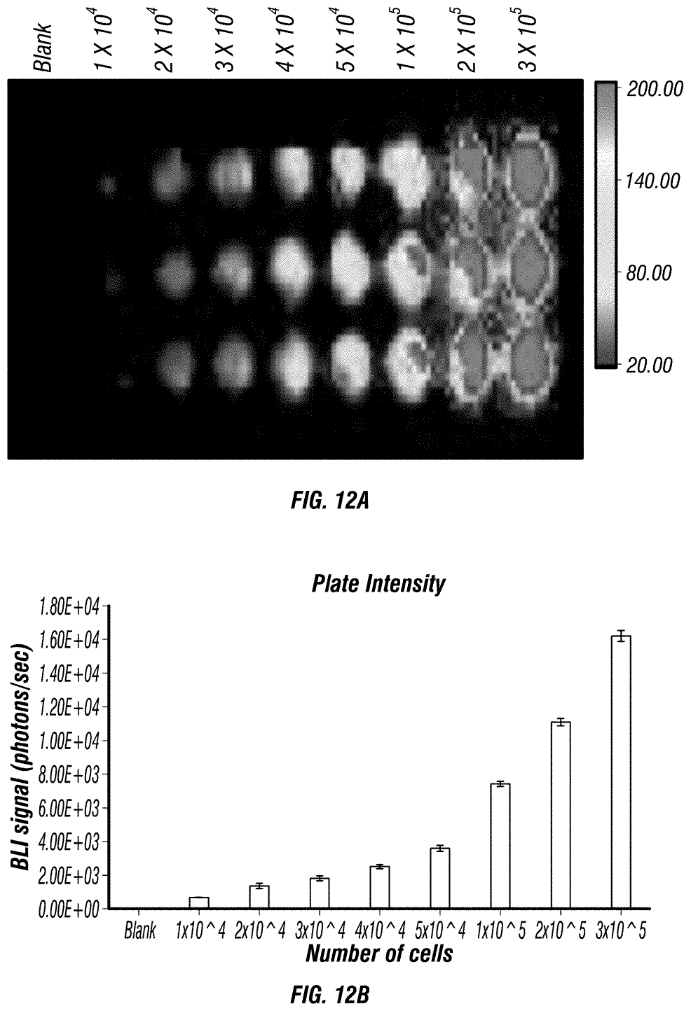

For selection, the cells were treated for 14 days with different concentrations of G418 antibiotic (0.1-0.8 mg/mL). After the antibiotic selection, the cells were screened for Luciferase expression using Steady-Glo Luciferase Assay System (Promega, USA). The U87MG cells treated with 0.8 mg/mL of G418 antibiotic was yielded the maximum luminescence. The luciferase transfection optical imaging made it possible to monitor response to anticancer therapies in tumor xenografts. In addition, luciferase images of the plated U87MG-luc cells showed a steady increase in the BLI signal as the number of cells increases (FIGS. 12A-B).

### Toxicity Evaluation

In order to evaluate the toxicity profile of F16, TMZ and F16+TMZ combination, a comprehensive toxicity study using BALB/c mice was performed. Mice injected with KP were used as controls. All drugs were administered as i.p. injections twice a week for 4 weeks. Independent toxicity evaluations, serum biochemistry, post-mortem gross examination, and histopathological examination of major organs were performed at Comparative Pathology Department at the University of Miami, Fla.

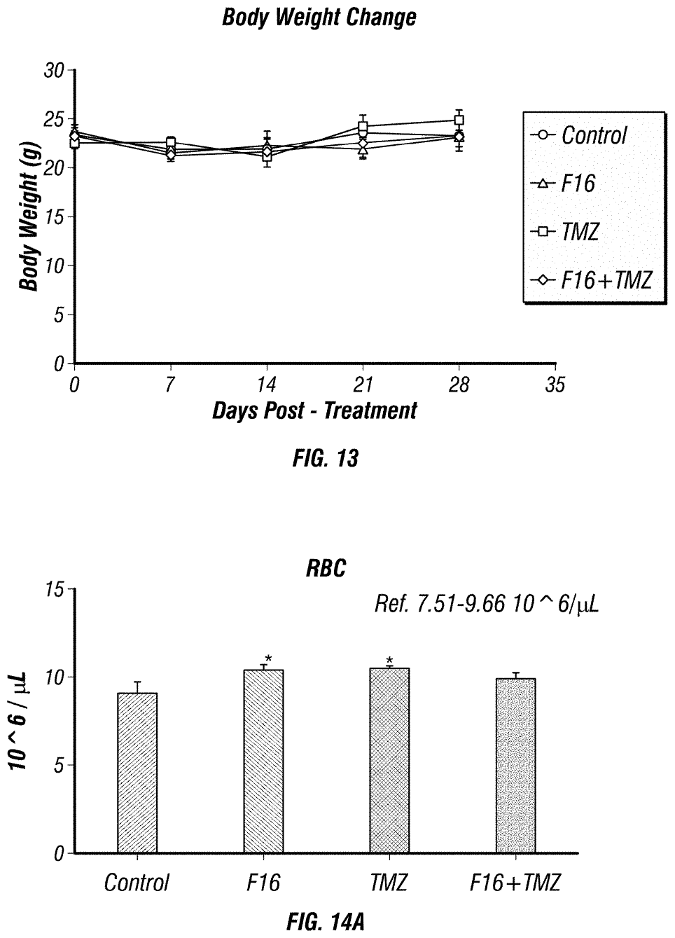

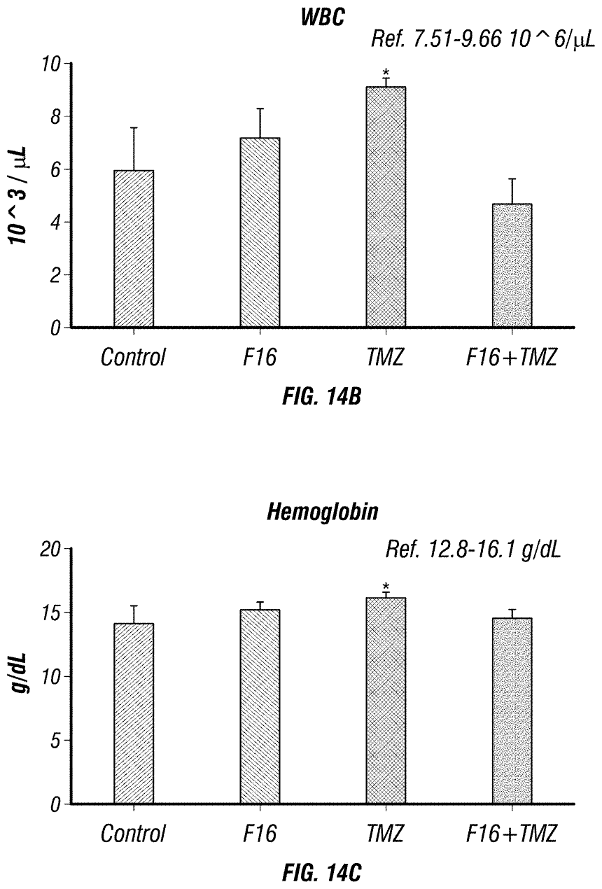

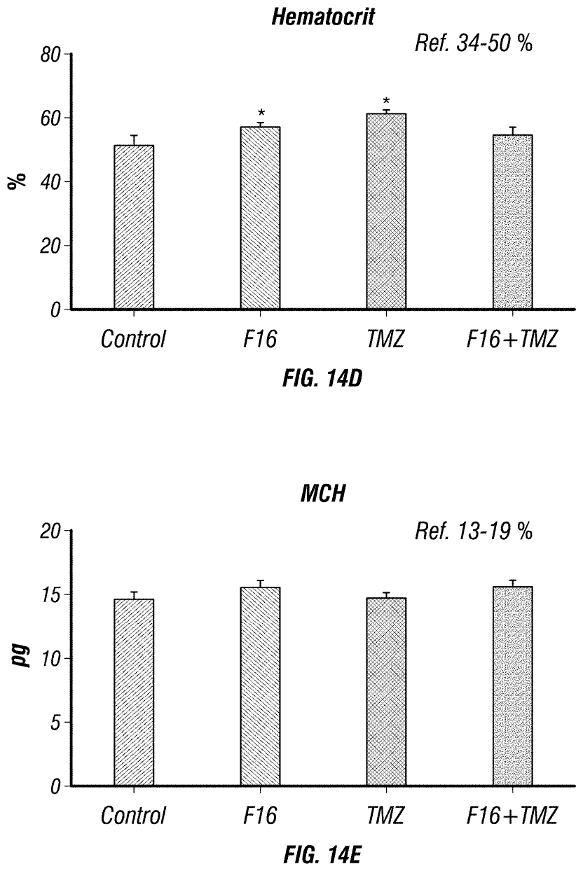

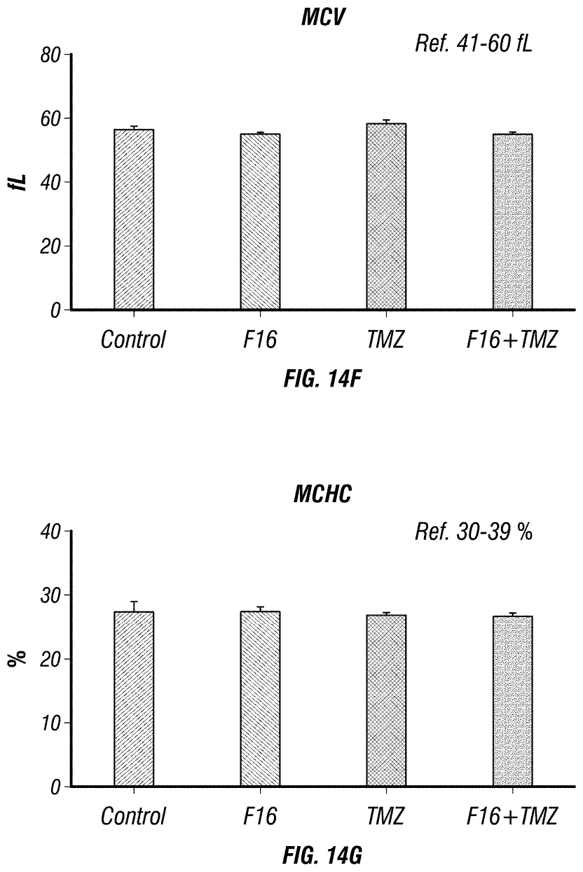

During the treatment period, changes in body weights of mice were checked weekly and no significant variation in the body weight was observed (FIG. 13) Also, mice were monitored for general behavioral, physical appearance, convulsions, drug-induced diarrhea, salivation, and mortalities. In general, F16 treatment was associated with no observable signs of toxicity. However, some symptoms of sensitivity or discomfort were noticed in the F16, combination and control (KP) groups immediately after giving the injection and then the symptoms disappeared in the next day. Since the same symptoms were seen in the control group, and F16 was well tolerated and no signs of toxicity or discomfort were observed in all previous animal experiments, KP is suspected as the reason of these symptoms.

Complete blood count (CBC) was performed to measure levels of hemoglobin (HB), hematocrit, mean corpuscular volume (MCV), mean corpuscular hemoglobin (MCH), mean corpuscular hemoglobin concentration (MCHC), red blood counts (RBC), and white blood cells counts (WBC). The levels of HB, MCH, MCHC, and MCV were not significantly altered in various treatment groups (FIG. 15: Table 1). A slight increase in hematocrit and RBC were observed in F16 and TMZ treated groups but not with KP and F16+TMZ treated groups (FIG. 15: Table 1). These results indicate no signs of bone marrow suppression leading to anemia, thrombocytopenia, or neutropenia (FIG. 15: Table 1). Analysis of WBC counts showed no significant changes in KP, F16, and F16+TMZ treated groups, however, TMZ treated mice showed a significant increase in WBC counts (FIG. 15: Table 1).

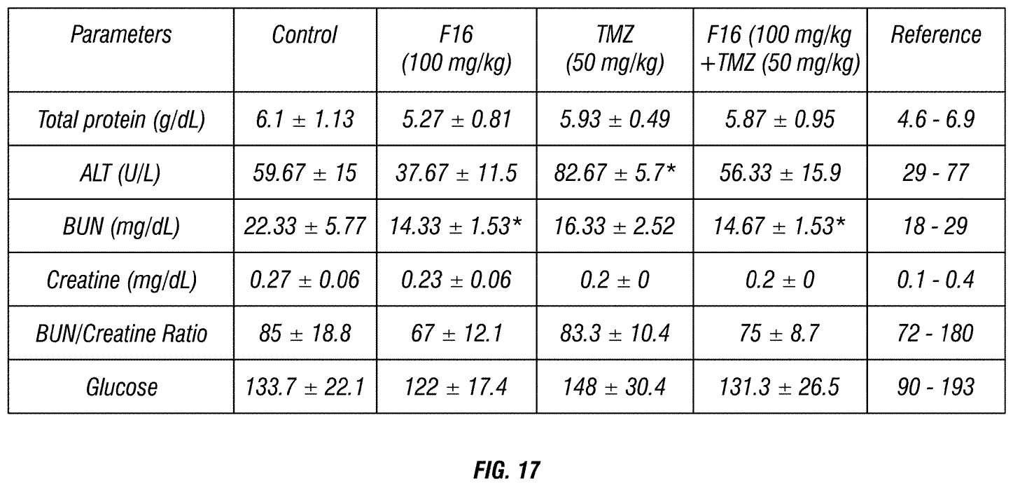

Total protein level was analyzed to evaluate the impact of treatment regimens on protein metabolism. No significant changes were observed in total protein levels in all treatment groups (FIG. 17: Table 2).

### Assessment of the Major Organ Function

Assessments of liver function were accomplished by measuring the level of ALT. Significant elevation of ALT level was observed in TMZ treated group (FIG. 17: Table 2). Furthermore, kidney function was evaluated by measuring the levels of blood urea nitrogen (BUN), creatine, and BUN/creatine ratio. No significant changes in BUN were detected in KP and TMZ treated groups. However, a significant reduction of BUN levels was observed in F16 and F16+TMZ treated groups (Table 2). As shown in FIG. 17 (Table 2), no significant change in the creatine and the ratio of BUN/creatine levels were found in all treatment groups. In addition, the effect of F16 on the pancreas was also evaluated by measuring glucose, an essential source of energy. No significant changes were observed in the blood-glucose in all treatment groups (FIG. 17: Table 2).

### Inhibition of U87MG Derived Xenograft Tumor Growth by F16

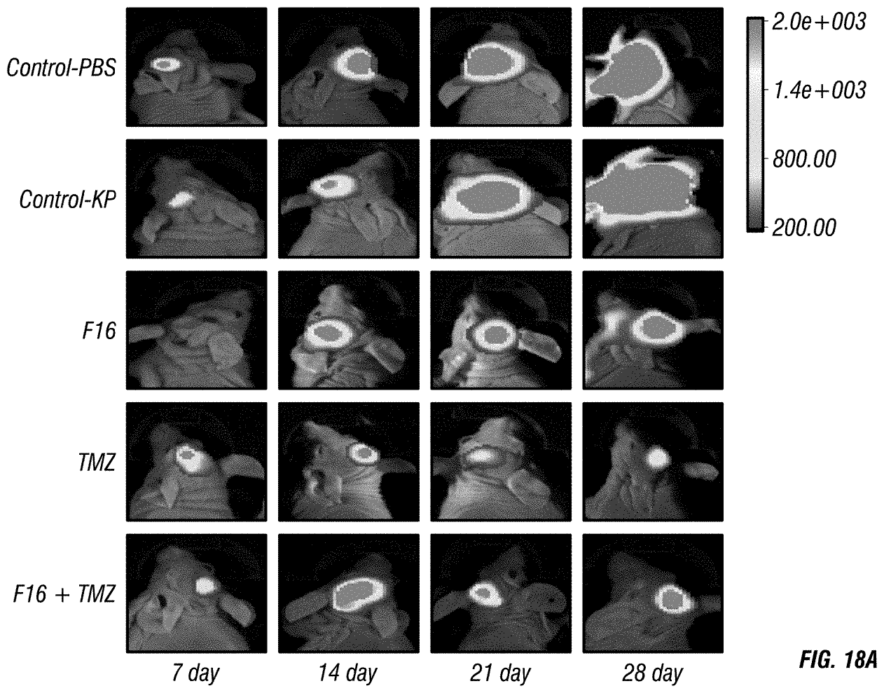

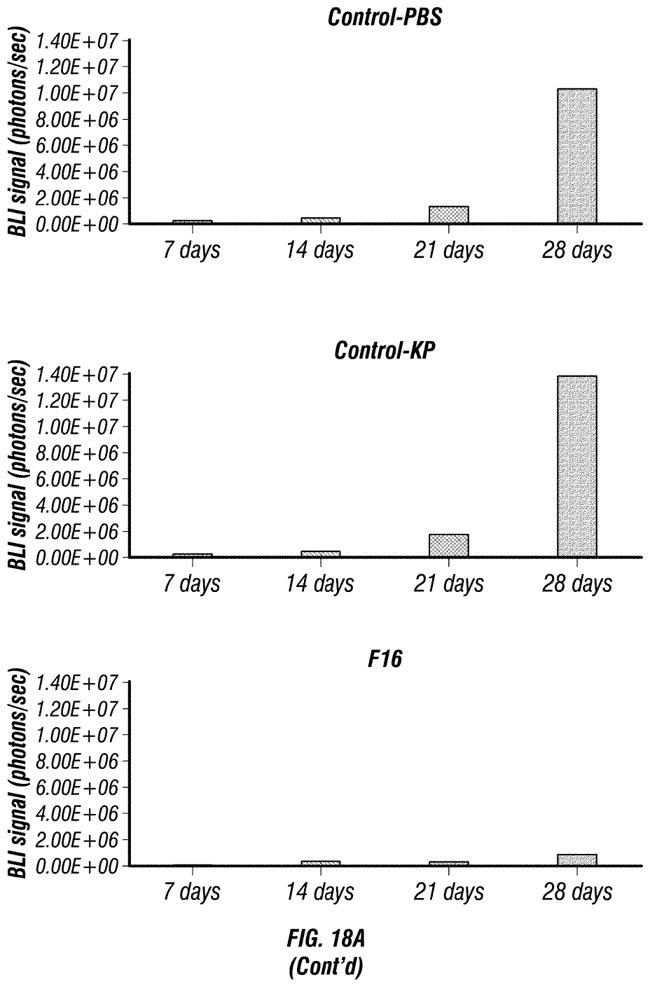

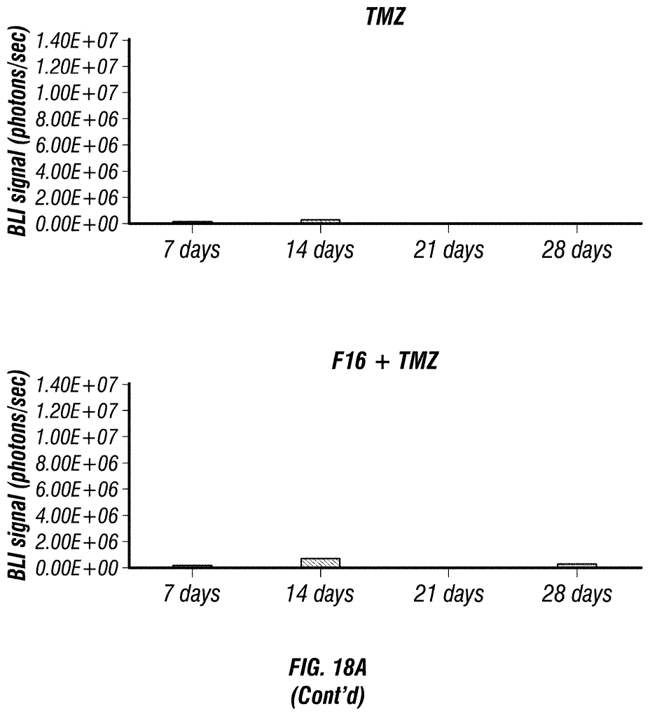

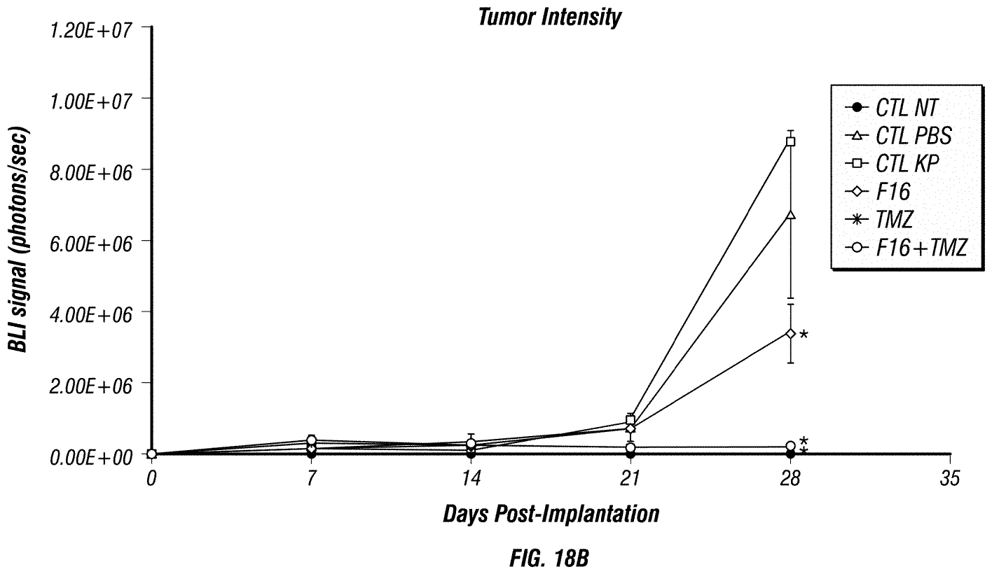

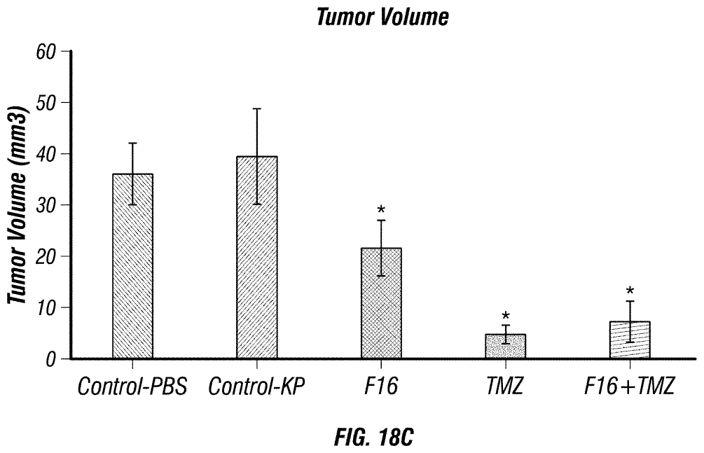

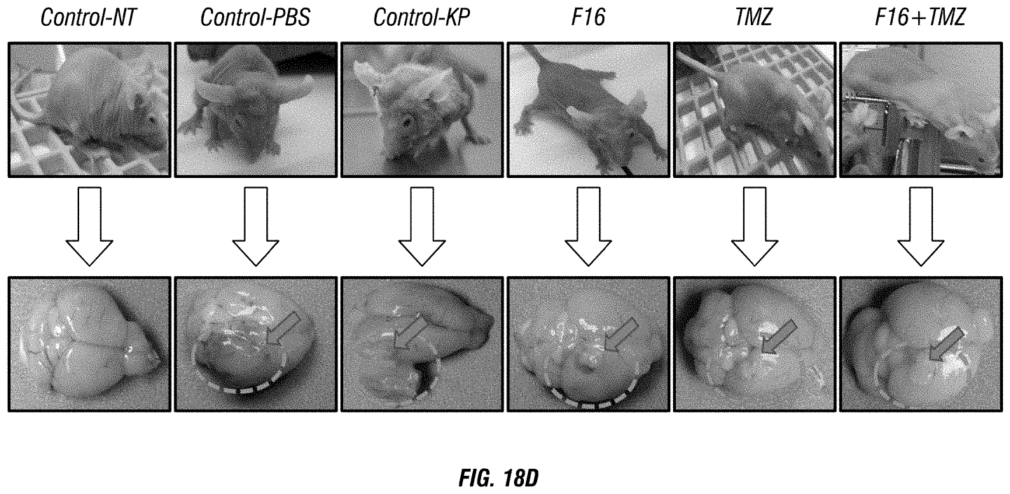

To further investigate the in vivo tumor growth inhibitory effects of F16 and to confirm the earlier study with the subcutaneous model, an intracranial glioblastoma xenograft model using U87MG cells was established as described earlier in the materials and methods section. U87MG-luc cells were implanted into the mice brains and tumor growth was monitored with BLI. One week after cell implantation, animals were randomly divided into five groups (control-PBS, control-KP, F16, TMZ, F16+TMZ). Tumor growth was monitored with BLI every week and representative mice from the five groups are shown in FIG. 18A. The results clearly showed that, the BLI signal intensity of F16 treated mice was 60% lower than control mice (FIG. 18B). However, the BLI signal intensity of the TMZ and combination treated mice were the lowest among the 5 groups (FIG. 18B). These results indicated that administration of F16 either monotherapy or combination decreased tumor growth; however, TMZ treatment was more efficient than F16 treatment which is expected since F16 is a cytostatic not cytoreductive. Moreover, after mice death, the brains with tumors were excised and then the brain tumor length (L) and width (W) were measured to calculate the tumor volume (TV) according to the formula: TV=1/2×(L×W²) (FIG. 18C). Representative images of athymic nude mice before euthanasia and excised brains with tumors from the same mice after euthanasia are shown in FIG. 18D.

### Survival Rate and Signs of Toxicity

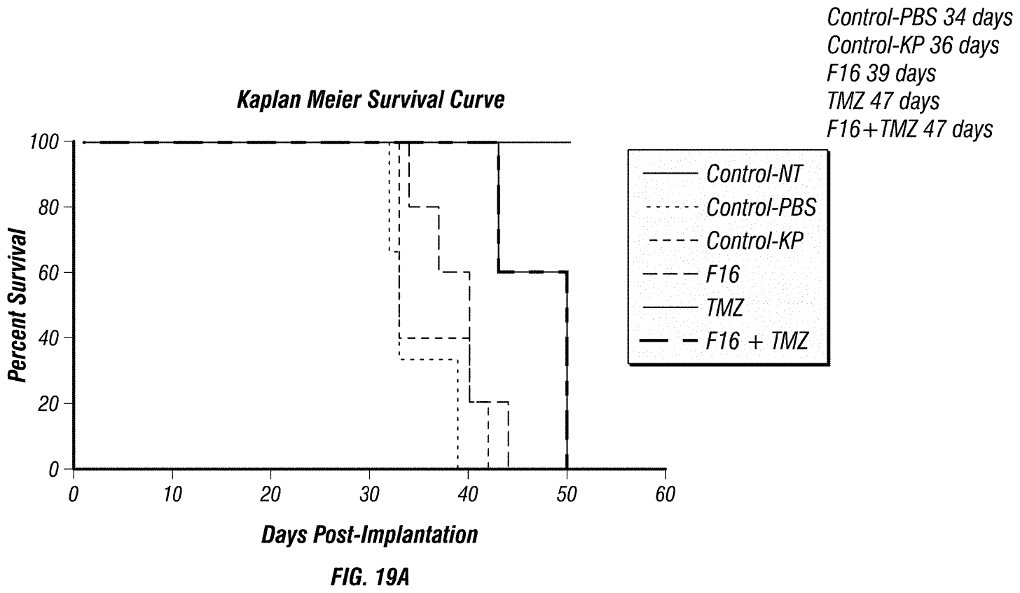

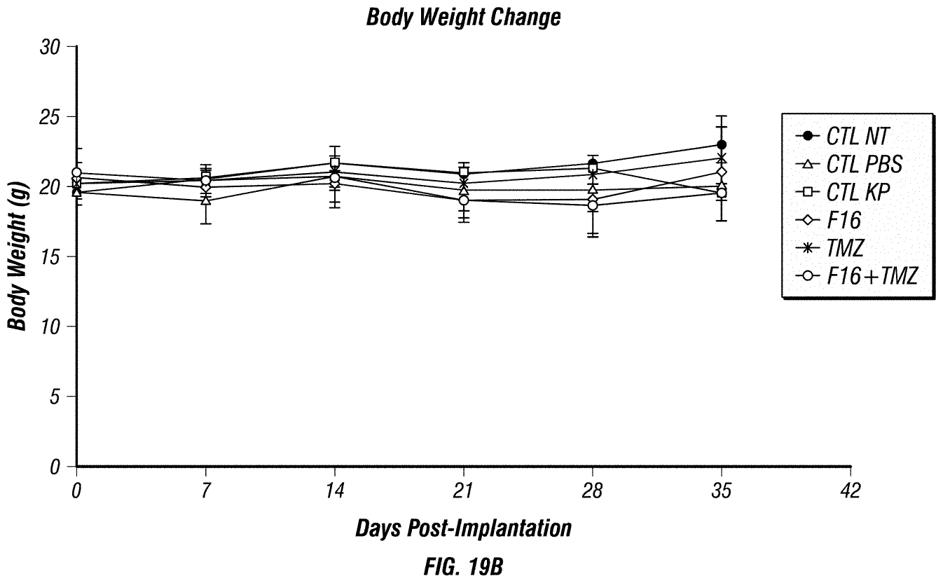

The survival of mice with glioma xenografts after vehicle-PBS, vehicle-KP, F16, TMZ and combination treatments was examined. Tumor bearing mice treated with F16 showed a significant increase in the survival time with a median survival of 39 days compare to mice treated with vehicles-PBS and vehicles-KP with a median survival of 34 days and 36 days respectively (FIG. 19A). Furthermore, 60% of mice in the TMZ and combination groups lived until day 50 post implantation with a median survival of 47 days (FIG. 19B). However, brains that were excised from TMZ and combination groups were fragile and damaged.

Changes in body weight of the experimental mice were examined weakly from the day of implantation until the end of the experiment (FIG. 19B). Consistent with previous experiments, F16 treatment was well tolerated at the dose that was used in the treatment (100 mg/kg). No significant change in body weight was observed in mice treated with F16, TMZ and combination compared to the mice treated with vehicles (PBS/KP).

### Microvessel Density Assessment

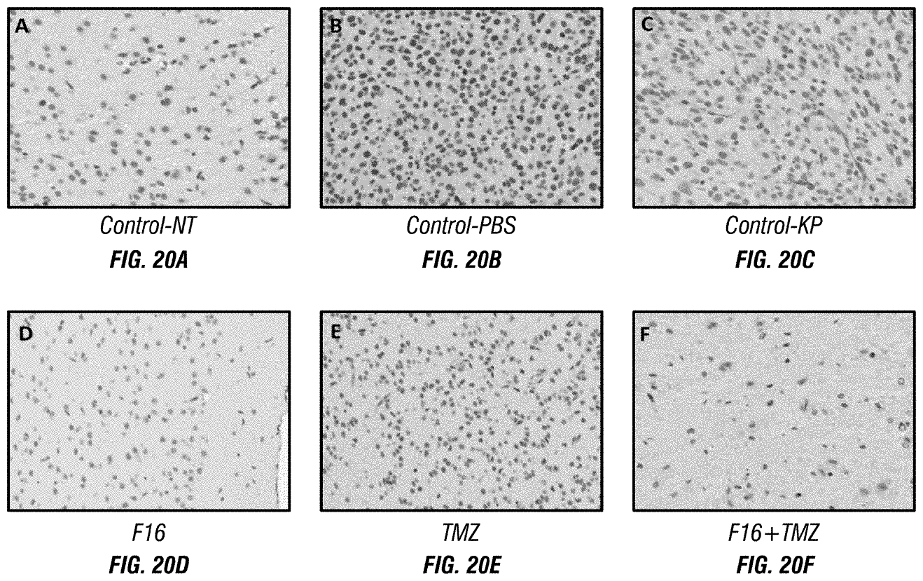

The xenograft brains and tumors were excised and subjected to IHC analysis. The expression of glioblastoma marker CD31 in F16, TMZ and combination of F16 and TMZ treated tumor section were compared to the tumors extracted from the control groups (FIGS. 20A-F). In the control-PBS and control-KP tumor sections, CD31 was expressed in high levels, which indicated that the exponential growth of GBM is associated with angiogenesis (FIGS. 20B-C). In contrast, a significant reduction of CD31 expression was observed in F16 tumor section compare to controls and TMZ tumor sections, indicating that F16 treatment effectively blocked angiogenesis in vivo (FIGS. 20D-E). This result showed that the reduction of vascular density was more prominent and informative of the anti-tumor activity of F16 exerted through reducing the vascular density of the xenograft tumor.

### Discussion

As noted above, Glioblastoma multiforme (GBM) treatment is very challenging as evidenced by the low survival rate of GBM patients, who generally do not live more than one year [22]. The current standard treatment for patients with GBM is multi-modal, which begins with extensive surgical resection of the tumor mass. Thereafter, patients are subjected to radiotherapy (RT) and concomitantly chemotherapy with Temozolomide (TMZ). Indeed, TMZ plus RT treatment regimen is considered to be the most effective as it increases the median overall survival by 2.6 months to be 14.6 months compared to RT alone 12 months, and the percentage of patients who live 2 years increases from 10.4% to 26.5% [4]. Unfortunately, 60-75% of TMZ treated patients do not respond to TMZ treatment and more than 50% of patients fail the treatment after 6 months of tumor progression [23, 24]. This lack of response is due to the over-expression of O⁶-methylguanine methyltransferase (MGMT) and/or DNA damage repair systems in GBM cells [25]. Moreover, 15-20% of TMZ treated patients develop significant toxicity, which can lead to disconsolation of treatment [23]. All these shortfalls associated with TMZ have promoted scientists to develop more effective therapeutic options. In this context, novel therapeutic strategies targeting vascular endothelial growth factor (VEGF) or its downstream signaling pathways have been yielding promising results as an addendum to standard therapy [26].

The dependence of tumor growth and metastasis on angiogenesis has supported the notion of using anti-angiogenic approaches in treating cancer. Moreover, angiogenesis inhibitors are clinically proven to improve patients' quality of life, extend progression free survival (PFS) and/or overall survival (OS) of several advanced stage cancers, which has prompted scientist to study using angiogenesis inhibitors for GBM treatment. In 2009, BVZ was approved by FDA for recurrent GBM treatment [27]. In fact, using BVZ for recurrent GBM treatment failed to improve the OS, but did improve the PFS [17, 28]. Moreover, angiogenesis inhibitors are proposed to be useful in alleviating the intracranial pressure associated with brain cancer by reducing the vessel permeability through normalization of the existing vasculature [29]. Unluckily, using angiogenesis inhibitors for GBM treatment is faced with two hurdles which are a few angiogenesis inhibitors can cross the blood brain barrier (BBB) [30], and some angiogenesis inhibitors associated with severe toxicities that limit their clinical benefits [31]. Therefore, there is a crucial need to develop novel angiogenesis inhibitors that can cross the BBB with little or no toxicity.

In 2011, F16, a novel antiangiogenic agent, was disclosed in U.S. Pat. No. 7,939,557 B2. F16 not only showed strong binding and inhibition of vascular endothelial growth factor receptor-2 (VEGFR2) phosphorylation in human umbilical vein endothelial cells (HUVEC) but also exhibited a significant in vivo tumor growth inhibition in mice implanted with GI-IOTA (breast cancer) xenograft and Colo-320 DM (colon cancer) xenograft [19]. In addition, the preclinical pharmacokinetic studies revealed substantial disposition of F16 in major organs of mice after a single i.p. administration [20]. It was an unexpected finding that F16 concentration at 12 h post injection was the highest in the brain compared to liver and kidneys. The concentration of F16 in the brain was close to the concentration that observed in the plasma, which was over 1.3 and 6.1 folds than liver and kidneys respectively. This result indicates that F16 is easily transported across the BBB and slowly accumulated into the brain regions without evidence of clinical behavioral toxicities. In fact, two important factors play a significant role in facilitating the BBB penetration of any drug, which are lipophilicity and molecular weight [32]. In consistent with these criteria, F16 is highly lipophilic an has a small molecular weight (301.2 g/mol), which may explain the penetration of the BBB. All these results inspired the inventors to test the effectiveness of F16 in the treatment of GBM.

Generally, treatment-related toxicity is one of the most common limitations of clinically available agents for cancer treatment. Hepatotoxicity and nephrotoxicity are the common toxicities associated with chemotherapeutic agents including angiogenesis inhibitors. In this toxicity study, mice treated with TMZ showed signs of liver toxicities as evidenced by the increase in the ALT (FIG. 17: Table 2). The results are also in agreement with a previous report of TMZ in rodent models [24]. In humans, TMZ treatment is associated with myelosuppression, including neutropenia and thrombocytopenia, in addition to the hepatotoxicity [33]. The previous results from the safety evaluation study have proven that F16 treated experimental animals remain healthy compared to the groups that were treated with other FDA approved chemo drugs such as Sutent® and Taxol [20]. Similarly, in the current study F16 was well-tolerated with no death events in experimental animals. Moreover, in the experimental groups, there was no significant change in body weight, food intake, or behavior (FIG. 13). Even though F16 was accumulated in the brain, no signs of cognitive changes were observed in the treatment groups. Furthermore, assessment of biochemical parameters that are reflecting vital organ functions showed no signs nor elevation in injury related biomarker levels of liver, kidney, and pancreases after F16 treatment.

Xenograft models using human cancer cells have provided tremendous benefits to oncology field. Initially, the subcutaneous xenograft model, which is called heterotopic, has been the most commonly used preclinical procedure to establish tumor xenografts because it is fast, inexpensive and easily reproducible [34, 35]. However, it has been consistently noticed that some drug regimens that are curative in heterotopic models do not have a significant effect on human disease. Therefore, the emphasis has been shifted towards orthotopic xenograft establishment such as intracranial brain tumor xenograft. In the orthotopic model, the tumor xenograft is implanted into the same anatomical location or organ from which the cancer is initiated, which will provide an appropriate location for tumor-host interactions, the ability to study the site-specific dependence of therapy and organ-specific expression of genes, and a sufficient preclinical test for anti-cancer drugs [35, 36]. Moreover, it is well known that tumor progression and metastasis are dependent on the formation of new blood vessels in most situations [37]. Also, the biochemical imbalance in the tumor microenvironment contributes to pathological angiogenesis and tumor growth progressions through continuous secretion of growth factors [38].

In order to mimic tumor growth with appropriate tumor microenvironment, the intracranial GBM xenograft model was established, which provides a better representation of the clinical features of tumor angiogenesis and be more relevant to the real situation inside the human brain. Results show that F16 significantly inhibited xenograft tumor growth (FIG. 18B), and prolonged the median survival (FIG. 19A), suggesting that VEGFR-2 blockade using F16 treatment is efficacious in delaying glioblastoma cancer growth. In the previous in vitro and in vivo (subcutaneous xenograft, Example 1) studies, F16 effect was comparable to TMZ effect. However, in intracranial xenograft model, TMZ showed much better tumor inhibition (99%) compared to F16 (60%), which is expected since F16 is cytostatic not cytoreductive as TMZ. Another possible reason behind the difference in the results is that the difference in the drug concentration that reach the brain after penetrating the BBB. All the drug concentration is reaching the cancer cell when in vitro model is used and substantial drug concentration is reaching the tumor site when subcutaneous xenograft model is used. On the contrary, drug delivery to the brain is influenced by several factor such as lipophilicity and small molecular weight due to the presence of the BBB. TMZ is able to cross the BBB easily because it is a lipophilic small molecule with a molecular weight of 194.15 g/mol [39]. Earlier study has used rats and monkeys to test the penetration of TMZ into the CNS and showed that the levels of TMZ in the brain are approximately 30-40% of the plasma concentration which is significant [40]. Undeniably, TMZ and combination treated groups lived longer than F16 treated group. However, brains that were excised from TMZ and combination groups were fragile and damaged, which imply that TMZ treatment is affecting the surrounding normal tissue and ultimately causing death. In agreement with our observation, a recent study concluded that TMZ treatment affects the extracellular matrix structure in normal brain tissue which might lead to the disease progression [41].

F16 is effectively mediated anti-tumor activity through inhibition of angiogenesis [19]. The IHC results confirmed the in vivo anti-angiogenic activity of F16 using CD31 expression as a biomarker to demonstrate the presence of endothelial cells in tumor tissues [42]. As expected, F16 treatment was associated with a low level of CD31 expression, representing a significant reduction of tumor micro-vessel density (FIG. 20D).

In conclusion to Example 2, the in vivo results clearly proved high potency of F16 treatment in inhibiting tumor growth and prolonging the median survival of mice implanting intracranially with U87MG-luc cells. In comparison to TMZ, F16 was well tolerated in mice without evidence of significant pre-clinical or laboratory toxicities. Though using KP formulation has improved the brain delivery of F16 by 40% compare to PBS formulation [data not shown], the KP formulation caused some hypersensitivity reactions which may lead to more serious side effect when it used for longer time [43]. Finally, these findings provide a new avenue for GBM treatment, which might benefit a significant number of patients by extending their overall survival or improve their quality of life.

### CONCLUSION

The findings disclosed herein provide a new avenue for treatment of solid cancers having angiogenic ability, particularly for treatments of brain cancers such as glioblastoma multiforme (GBM). Such novel treatments might benefit a significant number of patients by extending their overall survival and/or improve their quality of life.

All patents and publications mentioned in this specification are indicative of the levels of those skilled in the art to which the invention pertains. All patents and publications are herein incorporated by reference to the same extent as if each individual publication was specifically and individually indicated to be incorporated by reference. It is to be understood that while a certain form of the invention is illustrated, it is not intended to be limited to the specific form or arrangement herein described and shown. It will be apparent to those skilled in the art that various changes may be made without departing from the scope of the invention and the invention is not to be considered limited to what is shown and described in the specification. One skilled in the art will readily appreciate that the present invention is adapted to carry out the objectives and obtain the ends and advantages mentioned, as well as those inherent therein. The compositions and methods using F16 described herein are presently representative of the preferred embodiments, are intended to be exemplary and are not intended as limitations on the scope. Changes therein and other uses will occur to those skilled in the art which are encompassed within the spirit of the invention. Although the invention has been described in connection with specific, preferred embodiments, it should be understood that the invention as ultimately claimed should not be unduly limited to such specific embodiments. Indeed various modifications of the described modes for carrying out the invention which are obvious to those skilled in the art are intended to be within the scope of the invention.

### REFERENCES (ALL SECTIONS, EXCEPT EXAMPLE 2)

1. CBTRUS Fact Sheet. 2016—http://www.cbtrus.org/factsheet/factsheet.html.
2. Ostrom, Q. T., et al., American Brain Tumor Association Adolescent and Young Adult Primary Brain and Central Nervous System Tumors Diagnosed in the United States in 2008-2012. Neuro Oncol, 2016. 18 Suppl 1: p. i1-i50.
3. Jain, R. et al, Angiogenesis in brain tumours. Nat Rev Neurosci, 2007. 8(8): 610-22.
4. Kim, W. Y. and H. Y. Lee, Brain angiogenesis in developmental and pathological processes: mechanism and therapeutic intervention in brain tumors. FEBS J, 2009. 276(17): p. 4653-64.
5. Plate, K. H. and H. D. Mennel, Vascular morphology and angiogenesis in glial tumors. Experimental and Toxicologic Pathology, 1995. 47(2-3): p. 89-94.
6. K. Lamszus, P. K., M. Westphal, Invasion as limitation to anti-angiogenic glioma therapy. Acta Neurochir 2003. 88: p. pp 169-177.
7. Bullitt, E., D. A. Reardon, and J. K. Smith, A review of micro-and macrovascular analyses in the assessment of tumor-associated vasculature as visualized by MR. Neuroimage, 2007. 37: p. S116-S119.
8. E Taylor, T., F. B Furnari, and W. K Cavenee, Targeting EGFR for treatment of glioblastoma: molecular basis to overcome resistance. Current cancer drug targets, 2012. 12(3): p. 197-209.
9. Hurwitz, H., et al., Bevacizumab plus irinotecan, fluorouracil, and leucovorin for metastatic colorectal cancer. N Engl J Med, 2004. 350(23): p. 2335-42.
10. Sandler, A., et al., Paclitaxel-carboplatin alone or with bevacizumab for non-small-cell lung cancer. N Engl J Med, 2006. 355(24): p. 2542-50.
11. Miller, K., et al., Paclitaxel plus bevacizumab versus paclitaxel alone for metastatic breast cancer. N Engl J Med, 2007. 357(26): p. 2666-76.
12. Vincenzi, B., et al, Cetuximab and irinotecan as third-line therapy in advanced colorectal cancer patients: a single centre phase II trial. Br J Cancer, 2006. 94(6): p. 792-7.
13. Gerstner, E. R., et al., Phase I trial with biomarker studies of vatalanib (PTK787) in patients with newly diagnosed glioblastoma treated with enzyme inducing anti-epileptic drugs and standard radiation and temozolomide. J Neurooncol, 2011. 103(2): p. 325-32.
14. Drazin, D., et al., Long-term Remission Over Six Years for a Patient with Recurrent Glioblastoma Treated with Cediranib/Lomustine. Cureus, 2016. 8(1): p. e460.
15. Zustovich, F., et al., Sorafenib plus daily low-dose temozolomide for relapsed glioblastoma: a phase II study. Anticancer Res, 2013. 33(8): p. 3487-94.
16. Lakka, S. S. and J. S. Rao, Antiangiogenic therapy in brain tumors. Expert Rev Neurother, 2008. 8(10): p. 1457-73.
17. Friedman, H. S., et al., Bevacizumab alone and in combination with irinotecan in recurrent glioblastoma. Journal of clinical oncology, 2009. 27(28): p. 4733-4740.
18. Kreisl, T. N., et al., Phase II trial of single-agent bevacizumab followed by bevacizumab plus irinotecan at tumor progression in recurrent glioblastoma. Journal of clinical oncology, 2008. 27(5): p. 740-745.
19. Kerbel, R. and J. Folkman, Clinical translation of angiogenesis inhibitors. Nat Rev Cancer, 2002. 2(10): p. 727-39.
20. Vredenburgh, J. J., et al., Phase II trial of bevacizumab and irinotecan in recurrent malignant glioma. Clin Cancer Res, 2007. 13(4): p. 1253-9.
21. Nathanson, D. and P. S. Mischel, Charting the course across the blood-brain barrier. The Journal of clinical investigation, 2011. 121(1): p. 31.
22. Yamamoto, D., et al., Bevacizumab in the treatment of five patients with breast cancer and brain metastases: Japan Breast Cancer Research Network-07 trial. Onco Targets Ther, 2012. 5: p. 185-9.
23. Kazazi-Hyseni, F., J. H. Beijnen, and J. H. Schellens, Bevacizumab. Oncologist, 2010. 15(8): p. 819-25.
24. Rathinavelu, A., et al., Anti-cancer effects of F16: A novel vascular endothelial growth factor receptor-specific inhibitor. Tumour Biol, 2017. 39(11): p. 1010428317726841.
25. Alhazzani K, et al., Pharmacokinetic and safety profile of a novel anti-angiogenic agent F16 with high levels of distribution to the brain. American Association for Pharmaceutical Scientists (AAPS), 2016. Abstract-3312.
26. Laemmli, U. K., Cleavage of structural proteins during the assembly of the head of bacteriophage T4. nature, 1970. 227(5259): p. 680-685.
27. Wen, W., et al., Grape seed extract inhibits angiogenesis via suppression of the vascular endothelial growth factor receptor signaling pathway. Cancer Prev Res (Phila), 2008. 1(7): p. 554-61.
28. Jacobs, V. L., et al., Current review of in vivo GBM rodent models: emphasis on the CNS-1 tumour model. ASN Neuro, 2011. 3(3): p. e00063.
29. Clark, M. J., et al., U87MG decoded: the genomic sequence of a cytogenetically aberrant human cancer cell line. PLoS Genet, 2010. 6(1): p. e1000832.
30. Lee, C. Y., Strategies of temozolomide in future glioblastoma treatment. Onco Targets Ther, 2017. 10: p. 265-270.
31. Roger Stupp, M. D., et al, Radiotherapy plus Concomitant and Adjuvant Temozolomide for Glioblastoma. The new england journal of medicine, Mar. 10, 2005.
32. Neyns, B., et al., Dose-dense temozolomide regimens: antitumor activity, toxicity, and immunomodulatory effects. Cancer, 2010. 116(12): p. 2868-77.
33. Housman, G., et al., Drug resistance in cancer: an overview. Cancers (Basel), 2014. 6(3): p. 1769-92.
34. Szabo, E., et al., Autocrine VEGFR1 and VEGFR2 signaling promotes survival in human glioblastoma models in vitro and in vivo. Neuro-oncology, 2016. 18(9): p. 1242-1252.
35. Lee, S. Y., Temozolomide resistance in glioblastoma multiforme. Genes & Diseases, 2016. 3(3): p. 198-210.
36. Lan, F., et al., Sulforaphane reverses chemo-resistance to temozolomide in glioblastoma cells by NF-kappaB-dependent pathway downregulating MGMT expression. Int J Oncol, 2016. 48(2): p. 559-68.
37. Castro, G. N., et al., Effects of temozolomide (TMZ) on the expression and interaction of heat shock proteins (HSPs) and DNA repair proteins in human malignant glioma cells. Cell Stress Chaperones, 2015. 20(2): p. 253-65.
38. Baer, J. C., et al., Depletion of O6-alkylguanine-DNA alkyltransferase correlates with potentiation of temozolomide and CCNU toxicity in human tumour cells. Br J Cancer, 1993. 67(6): p. 1299-302.
39. Borowicz, S., et al., The soft agar colony formation assay. J Vis Exp, 2014(92): p. e51998.
40. Abhinand, C. S., et al., VEGF-A/VEGFR2 signaling network in endothelial cells relevant to angiogenesis. J Cell Commun Signal, 2016. 10(4): p. 347-354.
41. Wee, K. B. and B. D. Aguda, Akt versus p53 in a network of oncogenes and tumor suppressor genes regulating cell survival and death. Biophys J, 2006. 91(3): p. 857-65.
42. Franke, T. F., et al., PI3K/Akt and apoptosis: size matters. Oncogene, 2003. 22(56): p. 8983-98.
43. Gottlieb, T. M., et al., Cross-talk between Akt, p53 and Mdm2: possible implications for the regulation of apoptosis. Oncogene, 2002. 21(8): p. 1299-303.
44. Nakada, M., Y. Okada, and J. Yamashita, The role of matrix metalloproteinases in glioma invasion. Front Biosci, 2003. 8: p. e261-9.
45. Bernhart, E., et al., Protein kinase D2 regulates migration and invasion of U87MG glioblastoma cells in vitro. Exp Cell Res, 2013. 319(13): p. 2037-48.
46. Hagemann, C., et al., A complete compilation of matrix metalloproteinase expression in human malignant gliomas. World J Clin Oncol, 2012. 3(5): p. 67-79.
47. Hagemann, C., et al., Comparative expression pattern of Matrix-Metalloproteinases in human glioblastoma cell-lines and primary cultures. BMC Res Notes, 2010. 3: p. 293.
48. Chen, G., et al., Plumbagin suppresses the migration and invasion of glioma cells via downregulation of MAP-2/9 expression and inaction of PI3K/Akt signaling pathway in vitro. J Pharmacol Sci, 2017. 134(1): p. 59-67.
49. Li, C., et al., Sulforaphane inhibits invasion via activating ERK1/2 signaling in human glioblastoma U87MG and U373MG cells. PLoS One, 2014. 9(2): p. e90520.
50. Peng, X., et al., Sulforaphane inhibits invasion by phosphorylating ERK1/2 to regulate E-cadherin and CD44v6 in human prostate cancer DU145 cells. Oncol Rep, 2015. 34(3): p. 1565-72.
51. Marshall, C. J., Specificity of receptor tyrosine kinase signaling: transient versus sustained extracellular signal-regulated kinase activation. Cell, 1995. 80(2): p. 179-85.
52. Burotto, M., et al., The MAPK pathway across different malignancies: a new perspective. Cancer, 2014. 120(22): p. 3446-56.
53. Deschenes-Simard, X., et al., ERKs in cancer: friends or foes? Cancer Res, 2014. 74(2): p. 412-9.
54. Mebratu, Y. and Y. Tesfaigzi, How ERK1/2 activation controls cell proliferation and cell death: Is subcellular localization the answer? Cell Cycle, 2009. 8(8): p. 1168-75.
55. Yang, T. Y., et al., Sustained activation of ERK and Cdk2/cyclin-A signaling pathway by pemetrexed leading to S-phase arrest and apoptosis in human non-small cell lung cancer A549 cells. Eur J Pharmacol, 2011. 663(1-3): p. 17-26.

### REFERENCES (EXAMPLE 2)

1. Siegel, R. L., K. D. Miller, and A. Jemal, Cancer statistics, 2018. CA Cancer J Clin, 2018. 68(1): p. 7-30.
2. Kim, W. Y. and H. Y. Lee, Brain angiogenesis in developmental and pathological processes: mechanism and therapeutic intervention in brain tumors. FEBS J, 2009. 276(17): p. 4653-64.
3. Anjum, K., et al., Current status and future therapeutic perspectives of glioblastoma multiforme (GBM) therapy: A review. Biomed Pharmacother, 2017. 92: p. 681-689.
4. Stupp, R., et al., Radiotherapy plus concomitant and adjuvant temozolomide for glioblastoma. N Engl J Med, 2005. 352(10): p. 987-96.
5. Stupp, R., et al., Effects of radiotherapy with concomitant and adjuvant temozolomide versus radiotherapy alone on survival in glioblastoma in a randomised phase III study: 5-year analysis of the EORTC-NCIC trial. Lancet Oncol, 2009. 10(5): p. 459-66.
6. Neyns, B., et al., Dose-dense temozolomide regimens: antitumor activity, toxicity, and immunomodulatory effects. Cancer, 2010. 116(12): p. 2868-77.
7. Das, S. and P. A. Marsden, Angiogenesis in glioblastoma. N Engl J Med, 2013. 369(16): p. 1561-3.
8. Dimberg, A., The glioblastoma vasculature as a target for cancer therapy. Biochem Soc Trans, 2014. 42(6): p. 1647-52.
9. Chaudhry, I. H., et al., Vascular endothelial growth factor expression correlates with tumour grade and vascularity in gliomas. Histopathology, 2001. 39(4): p. 409-15.
10. Huang, H., et al., Expression of VEGF and its receptors in different brain tumors. Neurol Res, 2005. 27(4): p. 371-7.
11. Xu, C., X. Wu, and J. Zhu, VEGF promotes proliferation of human glioblastoma multiforme stem-like cells through VEGF receptor 2. ScientificWorldJournal, 2013. 2013: p. 417413.
12. Hurwitz, H., et al., Bevacizumab plus irinotecan, fluorouracil, and leucovorin for metastatic colorectal cancer. N Engl J Med, 2004. 350(23): p. 2335-42.
13. Sandler, A., et al., Paclitaxel-carboplatin alone or with bevacizumab for non-small-cell lung cancer. N Engl J Med, 2006. 355(24): p. 2542-50.
14. Miller, K., et al., Paclitaxel plus bevacizumab versus paclitaxel alone for metastatic breast cancer. N Engl J Med, 2007. 357(26): p. 2666-76.
15. Vincenzi, B., et al., Cetuximab and irinotecan as third-line therapy in advanced colorectal cancer patients: a single centre phase II trial. Br J Cancer, 2006. 94(6): p. 792-7.
16. Kreisl, T. N., et al., Phase II trial of single-agent bevacizumab followed by bevacizumab plus irinotecan at tumor progression in recurrent glioblastoma. Journal of clinical oncology, 2008. 27(5): p. 740-745.
17. Friedman, H. S., et al., Bevacizumab alone and in combination with irinotecan in recurrent glioblastoma. Journal of clinical oncology, 2009. 27(28): p. 4733-4740.
18. Weidle, U. H., J. Niewohner, and G. Tiefenthaler, The Blood-Brain Barrier Challenge for the Treatment of Brain Cancer, Secondary Brain Metastases, and Neurological Diseases. Cancer Genomics Proteomics, 2015. 12(4): p. 167-77.
19. Rathinavelu, A., et al., Anti-cancer effects of F16: A novel vascular endothelial growth factor receptor-specific inhibitor. Tumour Biol, 2017. 39(11): p. 1010428317726841.
20. Alhazzani K, et al., Pharmacokinetic and safety profile of a novel anti-angiogenic agent F16with high levels of distribution to the brain. American Association for Pharmaceutical Scientists (AAPS), 2016. Abstract-3312.
21. Strickley, R. G., Solubilizing excipients in oral and injectable formulations. Pharm Res, 2004. 21(2): p. 201-30.
22. deSouza, R. M., et al., Has the survival of patients with glioblastoma changed over the years? Br J Cancer, 2016. 114(2): p. 146-50.
23. Chamberlain, M. C., Temozolomide: therapeutic limitations in the treatment of adult high-grade gliomas. Expert Rev Neurother, 2010. 10(10): p. 1537-44.
24. Kim, S. S., et al., Encapsulation of temozolomide in a tumor-targeting nanocomplex enhances anti-cancer efficacy and reduces toxicity in a mouse model of glioblastoma. Cancer Lett, 2015. 369(1): p. 250-8.
25. Ramirez, Y. P., et al., Glioblastoma multiforme therapy and mechanisms of resistance. Pharmaceuticals (Basel), 2013. 6(12): p. 1475-506.
26. von Baumgarten, L., et al., Bevacizumab has differential and dose-dependent effects on glioma blood vessels and tumor cells. Clin Cancer Res, 2011. 17(19): p. 6192-205.
27. Castro, B. A. and M. K. Aghi, Bevacizumab for glioblastoma: current indications, surgical implications, and future directions. Neurosurg Focus, 2014. 37(6): p. E9.
28. Vredenburgh, J. J., et al., Phase II trial of bevacizumab and irinotecan in recurrent malignant glioma. Clin Cancer Res, 2007. 13(4): p. 1253-9.
29. Gerstner, E. R., et al., VEGF inhibitors in the treatment of cerebral edema inpatients with brain cancer. Nat Rev Clin Oncol, 2009. 6(4): p. 229-36.
30. Verhoeff, J. J., et al., Concerns about anti-angiogenic treatment in patients with glioblastoma multiforme. BMC Cancer, 2009. 9: p. 444.
31. Cheng, H. and T. Force, Molecular mechanisms of cardiovascular toxicity of targeted cancer therapeutics. Circ Res, 2010. 106(1): p. 21-34.
32. Banks, W. A., Characteristics of compounds that cross the blood-brain barrier. BMC Neurol, 2009. 9 Suppl 1: p. S3.
33. Sarganas, G., et al., Severe sustained cholestatic hepatitis following temozolomide in a patient with glioblastoma multiforme: case study and review of data from the FDA adverse event reporting system. Neuro Oncol, 2012. 14(5): p. 541-6.
34. Ozawa, T. and C. D. James, Establishing intracranial brain tumor xenografts with subsequent analysis of tumor growth and response to therapy using bioluminescence imaging. J Vis Exp, 2010(41).
35. Huynh, A. S., et al., Development of an orthotopic human pancreatic cancer xenograft model using ultrasound guided injection of cells. PLoS One, 2011. 6(5): p. e20330.
36. Huszthy, P. C., et al., In vivo models of primary brain tumors: pitfalls and perspectives. Neuro-oncology, 2012. 14(8): p. 979-993.
37. Folkman, J., Tumor angiogenesis: therapeutic implications. New england journal of medicine, 1971. 285(21): p. 1182-1186.
38. Nishida, N., et al., Angiogenesis in cancer. Vase Health Risk Manag, 2006. 2(3): p. 213-9.
39. Agarwala, S. S. and J. M. Kirkwood, Temozolomide, a novel alkylating agent with activity in the central nervous system, may improve the treatment of advanced metastatic melanoma. Oncologist, 2000. 5(2): p. 144-51.
40. Patel, M., et al., Plasma and cerebrospinal fluid pharmacokinetics of intravenous temozolomide in non-human primates. J Neurooncol, 2003. 61(3): p. 203-7.
41. Tsidulko, A. Y., et al., Conventional Anti-glioblastoma Chemotherapy Affects Proteoglycan Composition of Brain Extracellular Matrix in Rat Experimental Model in vivo. Front Pharmacol, 2018. 9: p. 1104.
42. Maj chrzak, K., et al., Markers of angiogenesis (CD31, CD34, rCBV) and their prognostic value in low-grade gliomas. Neurol Neurochir Pol, 2013. 47(4): p. 325-31.
43. Gelderblom, H., et al., Cremophor EL: the drawbacks and advantages of vehicle selection for drug formulation. Eur J Cancer, 2001. 37(13): p. 1590-8.

### Presentations

1. Mohammad Algahtanil, Khalid Alhazzani2, Thiagarajan Venkatesan, Ali Alaseem, Sivanesan Dhandayuthapani and Appu Rathinavelu (2019), Direct cytotoxic effect of a novel anti-angiogenic drug F16 towards U87MG glioblastoma cell line, Presented at the AACR Annual Meeting 2019, March 29-Apr. 3 Atlanta, Ga.
2. Mohammad Algahtani, Khalid Alhazzani, Sivanesan Dhandayuthapani, Thanigaivelan Kanagasabai, Appu Rathinavelu, (2017) F16 is a novel new candidate for brain tumors, Presented at Cancer Research and Targeted Therapy (CRT) Oct 26-28, Miami Fla., USA.
3. Sivanesan Dhandayuthapani, Thanigaivelan Kanagasabai, Khadija Cheema and Appu Rathinavelu (2017), Bioavailability, pharmacokinetics and safety profile of a novel anti-50 angiogenic compound JFD in pre-clinical models. Presented at the AACR Annual Meeting 2017, April 1-5 Washington, D.C.
4. Thanigaivelan Kanagasabai, Khalid Alhazzani, Thiagarajan Venkatesan, Sivanesan Dhandayuthapani, Ali Alaseem, Appu Rathinavelu (2017), impact of MDM2 inhibition on cell cycle regulation through Aurora Kinase B-CDK1 axis in prostate cancer cells, Presented at the Annual Conference of the American Association for Cancer Research (AACR) April 1-5, Washington, D.C., USA.
5. Ali Alaseem, Thiagarajan Venkatesan, Thanigaivelan Kanagasabai, Khalid Alhazzani, Saad Alobid, Priya Dondapati, Appu Rathinavelu (2017), increased MMPs activity in MDM2 overexpressing cancer cell lines, Presented at the Annual Conference of the American Association for Cancer Research (AACR) April 1-5, Washington, D.C., USA
6. Thiagarajan Venkatesan, Ali Alaseem, Khalid Alhazzani, Thanigaivelan Kanagasabai, Appu Rathinavelu (2017), Effects of histone deacetylase (HDAC) inhibitor on gene expression in MDM2 transfected prostate cancer cells, Presented at the Annual Conference of the American Association for Cancer Research (AACR) April 1-5, Washington, D.C., USA
7. Khalid Alhazzani, Ali Alaseem, Thiagarajan Venkatesan, Appu Rathinavelu (2017), Angiogenesis-related gene expression profile of a novel antiangiogenic agent F16 in human vascular endothelial cells, Presented at the Annual Conference of the American Association for Cancer Research (AACR) April 1-5, Washington, D.C., USA
8. Saad Ebrahim Alobid, Thiagarajan Venkatesan, Ali Alaseem, Khalid Alhazzani, Appu Rathinavelu (2017), analysis of human hypoxia related miRNA in MDM2 transfected prostate cancer cells, Presented at the Annual Conference of the American Association for Cancer Research (AACR) April 1-5, Washington, D.C., USA
9. Mohammad Algahtani, Khalid Alhazzani, Thiagarajan Venkatesan, Appu Rathinavelu (2017), apoptosis pathway-focused gene expression profiling of a novel VEGFR2 inhibitor, Presented at the Annual Conference of the American Association for Cancer Research (AACR) April 1-5, Washington, D.C., USA0.
10. Paramjot Kaur, Sivanesan Dhandayuthapani, Shona Joseph, Syed Hussain, Miroslav Gantar, Appu Rathinavelu. Evaluation of the cell surface binding of phycocyanin and associated mechanisms causing cell death in prostate cancer cells. Presented at the American Association for Cancer Research (AACR) 2017 Apr. 1-4; Washington D.C., USA
11. Khalid Alhazzani, Sivanesan Dhandayuthapani, Khadijah Cheema, Thanigaivelan Kanagasabai, Ali Alaseem, Thiagarajan Venkatesan, Appu Rathinavelu (2016), Pharmacokinetic and Safety Profile of a Novel Anti-angiogenic Agent F16 with High Levels of Distribution to the Brain. Presented in: 2016 AAPS Annual Meeting and Exposition at Colorado, Denver, on Nov. 16th 2016.
12. Thanigaivelan Kanagasabai, Sivanesan Dhandayuthapani, Khalid Alhazzani, Ali Alaseem and Appu Rathinavelu (2016), The pharmacodynamics profile and tissue distribution of a novel anti-angiogenic compound JFD in pre-clinical models. Presented in: Molecular and Cellular Basis of Breast Cancer Risk and Prevention at Tampa, Fla. on Nov., 12th-15th 2016.

### 13. Appu Rathinavelu (2016), Novel VEGFR2 Inhibitors for Treating Solid Tumors and Brain Metastasis (2016), Presented at the International Conference on Cancer Research and Targeted Therapy, in Baltimore, Md. on Oct. 21-23 of 2016.

14. Thanigaivelan Kanagasabai, Rohin Chand, Amy Aman Kaur, Sivanesan Dhandayuthapani, Olena Bracho, Appu Rathinavelu. MDM2 stabilizes and induces HIF-lu levels during reoxygenation of cancer cells. Presented at the Annual Conference of the American Association for Cancer Research (AACR), April 16-20, New Orleans, La., USA
15. Thiagarajan Venkatesan, Ali Alaseem, Aiyavu Chinnaiyan, Sivanesan Dhandayuthapani, Thanigaivelan Kanagasabai, Khalid Alhazzani, Priya Dondapati, Saad Alobid, Umamaheswari Natarajan, Ruben Schwartz, Appu Rathinavelu (2018). MDM2 Overexpression Modulates the Angiogenesis-Related Gene Expression Profile of Prostate Cancer Cells. Cells, 2018, 7(5), 41.
16. Appu Rathinavelu, Thanigaivelan Kanagasabai, Sivanesan Dhandayuthapani, Khalid Alhazzani (2018), The anti-angiogenic and pro-apoptotic effects of a small molecule JFD-WS in in vitro and breast cancer xenograft mouse model. Oncology Reports. Published online on: Feb. 9, 2018, Pages:1711-1724; https://doi.org/10.3892/or.2018.6256
17. Rathinavelu. A, Alhazzani. K, Dhandayuthapani. S and Kanagasabai. T. (2017) Anti-cancer effects of F16—A novel vascular endothelial growth factor receptor specific inhibitor, Tumor Biology, November; 39 (11):1010428317726841. https://doi: 10.1177/1010428317726841.

What is claimed is:

1. A composition for treatment of a solid tumor comprising F16.

2. The composition according to claim 1, wherein the solid tumor has angiogenic ability.

3. The composition according to claim 1, wherein the solid tumor is a brain cancer.

4. The composition according to claim 2, wherein the solid tumor is a brain cancer.

5. The composition according to claim 3 or claim 4, wherein the brain cancer is glioblastoma multiforme (GBM).

6. A pharmaceutical composition for treatment of a solid tumor comprising a therapeutically effective dosage of F16 in a pharmaceutical carrier.

7. The pharmaceutical composition according to claim 6, wherein the solid tumor has angiogenic ability.

8. The pharmaceutical composition according to claim 6 or claim 7, further comprising a therapeutically effective dosage of a chemotherapeutic agent.

9. The pharmaceutical composition according to claim 8, wherein the chemotherapeutic agent is temozolomide (TMZ) or bevacizumab (BVZ) or similar agents.

10. A pharmaceutical composition for treatment of brain cancer comprising a therapeutically effective dosage of F16 in a pharmaceutical carrier.

11. A pharmaceutical composition for treatment of glioblastoma multiforme (GBM) comprising a therapeutically effective dosage of F16 in a pharmaceutical carrier.

12. The pharmaceutical composition according to claim 10 or claim 11, further comprising a therapeutically effective dosage of a chemotherapeutic agent.

13. The pharmaceutical composition according to claim 12, wherein the chemotherapeutic agent is temozolomide (TMZ) or bevacizumab (BVZ) or similar agents.

14. A method for inhibiting vascular endothelial growth factor receptor-2 (VEGFR-2) in malignant cells, the method comprising:
providing a composition including F16; and
administering the composition to the malignant cells.

15. A method for inhibiting angiogenesis in tissue exhibiting aberrant vasculature, the method comprising:
providing a composition including F16; and
administering the composition to the tissue exhibiting aberrant vasculature.

16. A method for inhibiting phosphorylation of vascular endothelial growth factor receptor-2 (VEGFR-2) in malignant cells, the method comprising:
providing a composition including F16; and
administering the composition to the malignant cells.

17. A method for inhibiting invasion and migration of malignant cells into surrounding tissues, the method comprising:
providing a composition including F16; and
administering the composition to the malignant cells.

18. A method for inhibiting a cell cycle or inducing cell cycle arrest in malignant cells, the method comprising:
providing a composition including F16; and
administering the composition to the malignant cells.

19. A method for inducing apoptosis in malignant cells, the method comprising: providing a composition including F16; and
administering the composition to the malignant cells.

20. A method for treating brain cancer in a subject in need thereof by inhibiting vascular endothelial growth factor receptor-2 (VEGFR-2) in malignant cells of the brain cancer, the method comprising:
providing a composition including F16; and
administering the composition to the malignant cells of the brain cancer.

21. A method for treating brain cancer in a subject in need thereof by inhibiting angiogenesis in malignant cells of the brain cancer, the method comprising:
providing a composition including F16; and
administering the composition to the malignant cells of the brain cancer.

22. A method for treating brain cancer in a subject in need thereof by inhibiting phosphorylation of vascular endothelial growth factor receptor-2 (VEGFR-2) in malignant cells of the brain cancer, the method comprising:
providing a composition including F16; and
administering the composition to the malignant cells of the brain cancer.

23. A method for treating brain cancer in a subject in need thereof by inhibiting invasion and migration of malignant cells of the brain cancer into surrounding tissues, the method comprising:
providing a composition including F16; and
administering the composition to the brain cancer cells.

24. A method for treating brain cancer in a subject in need thereof by inhibiting a cell cycle or inducing cell cycle arrest in malignant cells of the brain cancer, the method comprising:
providing a composition including F16; and
administering the composition to the brain cancer cells.

25. A method for treating brain cancer in a subject in need thereof by inducing apoptosis in malignant cells of the brain cancer, the method comprising:
providing a composition including F16; and
administering the composition to the brain cancer cells.

26. The method of any of claims 20-25, wherein the brain cancer is glioblastoma multiforme (GBM).

27. A method for treating glioblastoma multiforme (GBM) in a subject in need thereof, the method comprising:
providing a composition including F16; and
administering the composition to the subject.

28. The method of any claims 20-27, wherein the composition provided further comprises a chemotherapeutic agent.

29. The method of claim 28, wherein the chemotherapeutic agent is temozolomide (TMZ) or bevacizumab (BVZ) or similar agents.

30. The pharmaceutical composition of any of claims 6-13 for use in a method for inhibiting vascular endothelial growth factor receptor-2 (VEGFR-2) in malignant cells.

31. The pharmaceutical composition of any of claims 6-13 for use in a method for inhibiting angiogenesis in tissue exhibiting aberrant vasculature.

32. The pharmaceutical composition of any of claims 6-13 for use in a method for inhibiting phosphorylation of vascular endothelial growth factor receptor-2 (VEGFR-2) in malignant cells.

33. The pharmaceutical composition of any of claims 6-13 for use in a method for inhibiting invasion and migration of malignant cells into surrounding tissues.

34. The pharmaceutical composition of any of claims 6-13 for use in a method for inhibiting a cell cycle or inducing cell cycle arrest in malignant cells.

35. The pharmaceutical composition of any of claims 6-13 for use in a method for inducing apoptosis in malignant cells.

36. The pharmaceutical composition of any of claims 6-13 for use in a method for treating glioblastoma multiforme (GBM) in a subject in need thereof.

37. The pharmaceutical composition of any of claims 6-13 for use in a method for treating brain cancer in a subject in need thereof by inhibiting vascular endothelial growth factor receptor-2 (VEGFR-2) in malignant cells of the brain cancer.

38. The pharmaceutical composition of any of claims 6-13 for use in a method for treating brain cancer in a subject in need thereof by inhibiting angiogenesis in malignant cells of the brain cancer.

39. The pharmaceutical composition of any of claims 6-13 for use in a method for treating brain cancer in a subject in need thereof by inhibiting phosphorylation of vascular endothelial growth factor receptor-2 (VEGFR-2) in malignant cells of the brain cancer.

40. The pharmaceutical composition of any one of claims 6-13 for use in a method for treating brain cancer in a subject in need thereof by inhibiting invasion and migration of malignant cells of the brain cancer into surrounding tissues.

41. The pharmaceutical composition of any of claims 6-13 for use in a method for treating brain cancer in a subject in need thereof by inhibiting a cell cycle or inducing cell cycle arrest in malignant cells of the brain cancer.

42. The pharmaceutical composition of any one of claims 6-13 for use in a method for treating brain cancer in a subject in need thereof by inducing apoptosis in malignant cells of the brain cancer.

43. Use of the pharmaceutical composition of any one of claims 37-42, wherein the brain cancer is glioblastoma multiforme (GBM).

44. Use of the pharmaceutical compositions of any one of claims 36-42, wherein the pharmaceutical composition further comprises a chemotherapeutic agent.

45. Use of the pharmaceutical composition of claim 44, wherein the chemotherapeutic agent is temozolomide (TMZ) or bevacizumab (BVZ) or similar agents.
